(19) United States
(12) Patent Application Publication
(10) Pub. No.: US 2025/0120624 A1
(43) Pub. Date: Apr. 17, 2025

(54) SYSTEM AND METHOD FOR OPTIMIZING PRECISION OF DIAGNOSTIC AND THERAPEUTIC PROCESSES

(71) Applicant: Listen Therapeutics Inc., New York, NY (US)

(72) Inventors: Eldad ELNEKAVE, Raanana (IL); Gilad MAROM, Magal (IL); Eran OREN, Tel Aviv (IL); Honorata KAZIMIERCZAK, Warsaw (PL)

(73) Assignee: Listen Therapeutics Inc., New York, NY (US)

(21) Appl. No.: 18/959,703

(22) Filed: Nov. 26, 2024

Related U.S. Application Data
(63) Continuation-in-part of application No. 18/854,984, filed on Jan. 1, 0001.
(60) Provisional application No. 63/329,978, filed on Apr. 12, 2022.

(51) Int. Cl. A61B 5/1473; A61B 5/15; A61M 5/172; G16H 20/40

(52) U.S. Cl. CPC A61B 5/1473; A61B 5/150992; A61M 5/1723; G16H 20/40

(57) ABSTRACT

The present invention discloses a system and method for optimizing diagnostic and therapeutic effects, and comprises diagnostic endovascular invasive device having sensing means and designated to be in a proximity to a target tissue in order to monitor and provide valuable data regarding its biochemical and/or physiological state in vivo and a controller designated to process data received from said sensors in order to produce medical recommendations and/or conclusions.

### CROSS-REFERENCE TO RELATED APPLICATIONS

This application is a continuation in part application of U.S. patent application Ser. No. 18/854,984, filed on Oct. 8, 2024, which is a National Phase Application of PCT International Application No. PCT/IL2023/050389, International Filing Date: Apr. 12, 2023, claiming priority of U.S. Provisional Patent Application No. 63/329,978, filed Apr. 12, 2022, which are hereby incorporated by reference.

### FIELD OF THE INVENTION

The present disclosure relates to means for optimizing therapeutic efficacy and particularly, but not exclusively, to systems and methods for optimizing treatment protocols' therapeutic effects, patient safety, treatment cost or any combination thereof by utilizing a diagnostic catheter-based device.

### BACKGROUND OF THE INVENTION

Cancer remains a leading cause of suffering and mortality worldwide. Systemic treatment options for cancer include traditional cytotoxic chemotherapy, targeted small molecules and biologic therapies including Immunotherapeutics and others. Patients with malignancy will typically undergo multiple successive lines of therapy over the course of years and few guidelines exist to inform the selection or duration of treatment with any one of multiple potential systemic options. The breadth of treatment options grows yearly with the introduction of new drugs, new classes of drugs and new combinations of existing drugs.

The match of drug to patient often begins with a biopsy. Biopsy is most commonly a sample of tissue or cells taken from the disease site and is assessed on several levels. Most basically, the fundamental histology is assessed. For example, a biopsy taken from a cancerous site and its surrounding is often informative regarding, e.g. the cells of origin, how rapidly are they proliferating, and whether they have extended into lymphatic/neural/vascular structures. By sequencing genomic features of the cells, particular mutations can be determined, which may be associated with prognostic information or predictive of therapeutic response to a given treatment. Surface cell staining may indicate what proteins are actually produced and expressed in the diseased site. The information extracted from a biopsy has some value in predicting which of the various courses of treatment are likely to be effective.

The abovementioned limitations of biopsies or other monitoring techniques in the state of the art, does not currently allow for reliable assessment of the treatment's efficacy and safety.

U.S. Pat. No. 7,787,937B2 discloses means of monitoring the progression of oncological disease and the assessment of treatment response by using an internal in-vivo sensor to detect radiation and wirelessly transmit information regarding the location of a radio-labeled compound injected into a tumor.

US20210207223A1 discloses a method configured to genetically monitor tumors by blood monitoring of cell-free DNA. U.S. Pat. No. 7,949,474B2 discloses a method that enables in-vivo microscopy for detecting cellular changes indicative of cancer-related mutations or the response to drugs. U.S. Pat. No. 9,789,241B2 discloses a system to monitor blood perfusion into an organ for facilitating cancer treatment.

Beyond the tools for tumor analyses and the trials to monitor diseases progression and response, the art has further shown some publications disclosing in-vivo measurements that range from pH sensing such as in U.S. Pat. No. 8,062,234B2, CN109289112B; publications disclosing spectroscopic measurements such as in US20150157405A1, U.S. Pat. No. 8,571,640B2 and publications disclosing biological sensing and assays such as in U.S. Pat. Nos. 9,119,533B2, 9,510,780B2.

However, none of the above publications discloses a venous diagnostic endovascular device configured to monitor a target tissue such as a tumor and provide valuable data regarding its biochemical and/or physiological state in vivo, wherein the configuration also prevents biochemical signals from being diluted or degraded in the blood stream before being detected.

Moreover, none of the above publications discloses combining the venous diagnostic endovascular device with an arterial endovascular catheter to achieve a complex system of real-time therapeutic and diagnostic feedback, as broadly disclosed below.

There are known companion diagnostic techniques that aim to narrow the gap between the growing numbers of therapeutic options on the one hand and the increasingly sophisticated sub-classifications of tumors and patients, on the other. Nevertheless, to date, predictions of therapy efficacy are severely limited. Tumor characterization is prone to sample error due to spatial tissue heterogeneity. Furthermore, a tumor sample is further disconnected from the relevant interactions of the host (for example, the immune system and microbiome) and from its immediate microenvironment. Compounding the limitation of spatial heterogeneity causes a high degree of temporal variation. In other words, tumors change quickly, due to mutagenesis that is itself influenced by therapeutic interventions.

Even reliable diagnostic tests cannot inform the appropriate dose or duration of treatment. Indications of treatment response and decisions to change drugs are generally based upon imaging features (for example, CT/PET/MRI) acquired in intervals of three to six months. If a new drug is introduced, it is generally chosen based upon the same diagnostic data available from the initiation of therapy. In essence, despite the dynamic nature of the disease, much of cancer treatment is based on long-term continuous therapy with few interspersed points of feedback.

However, as does any physiologic tissue, cancer provides continuous molecular feedback in response to stimuli, including stimuli from therapy. Incoming stimuli always travel to tissue in arteries whereas the resulting molecular feedback is released into the venous drainage from that tissue. Molecular products are most highly concentrated at the vein most proximal to the cells releasing those products.

Dilution occurs at every venous confluence as venous blood travel distally. In addition to the effect of spatial dilution, molecules undergo enzymatic degradation in short order once in the bloodstream. This physiologic principle underlies the use of selective venous blood sampling in order to localize the source of abnormal tissue-as in the case of hyperparathyroidism, adrenal adenoma or neuroendocrine tumors for example.

### SUMMARY OF THE INVENTION

The invention herein is a system and methods for data collection as part of a medical procedure configured to allow better insight into tumors, or other disease's response to therapy.

Biopsies are performed commonly and repeatedly in some patients, for diagnoses, severity or malignance assessment or treatment customization. They may be analyzed by a pathologist, by microscopically observing cell size, shape, tissue behavior or chemical compound analyses. However, the realistic time constraints are limiting the direct use of biopsies during medical procedures. In addition, by the nature of biopsies, they only show limited information representing a point in time and space, and cannot reflect holistic relevant information, such as a patient's microbiome or the microbiome of a tumor. As are the malignant cells of a tumor, so is its microenvironment heterogeneous and only partially sampled in a biopsy. Furthermore, physiological and biochemical characteristics change dramatically at different times within tumors, often in response to therapy.

According to some embodiment of the invention, at least one diagnostic catheter-based device is designated to be positioned in a draining vein proximal to a tumor or other tissue undergoing therapy. According to some embodiments, the diagnostic catheter-based device is simultaneously introduced with an arterial catheter capable of selectively infusing the tumor/target tissue of interest.

According to some embodiments, while the therapeutic arterial catheter is introducing an active material, the at least one diagnostic catheter monitors the biological response and reports physiological results and observations to medical practitioners. By utilizing and implementing the insight, the treatment efficacy may be improved, and safety issues may dealt with better.

According to some embodiments, the invention herein discloses the use of being in the close proximity of a tumor, and the use of diagnostic tools in parallel to the therapeutic effect, thus allowing data acquisition that would have otherwise been impossible to discern above the noise level of background physiology.

The invention further shows a plurality of measurement systems and techniques in the context of diagnostic catheters that achieve similar goals.

According to one aspect, there is provided a medical system comprising (i) at least one diagnostic endovascular invasive device designated to be inserted into the venous vasculature, (ii) at least one sensor integrated with the at least one diagnostic endovascular invasive device and configured to detect biochemical or physiological changes in a bodily fluid, (iii) a controller designated to receive data derived from the at least one sensor, wherein the at least one diagnostic endovascular invasive device is designated to be in a proximity to a target tissue, and wherein the controller is designated to process received data in order to produce medical recommendations and/or conclusions.

According to some embodiments, the target tissue is a tumor and wherein the diagnostic endovascular invasive device is designated to monitor biochemical or physiological changes in a blood stream originating from the tumor.

According to some embodiments, the diagnostic endovascular invasive device comprises at least two lumens, wherein the first lumen comprises at least one sensor and the second lumen comprises operational means.

According to some embodiments, the second lumen comprises openings configured to infuse designated substance/s having an effect on the fluid surrounding the at least one sensor in the first lumen.

According to some embodiments, the infused substance/s is designated to prevent blood clots in the proximity of the at least one sensor.

According to some embodiments, the infused substance/s is designated to improve the sensing capabilities of the at least one sensor.

According to some embodiments, the system further comprises visual internal control means designated to provide spectroscopic control reference to the at least one sensor.

According to some embodiments, at least two sensors are positioned in a certain distance from each other along the diagnostic endovascular invasive device.

According to some embodiments, at least two sensors are arranged in a staggered way geometrically and are configured to deduce spatial and spatiotemporal information regarding biochemical or physiological signals found in proximity to the diagnostic endovascular invasive device.

According to some embodiments, the system further comprising a therapeutic endovascular invasive device designated to be inserted into the arterial vasculature and administrate a designated substance/s in order to affect the at least one sensor of the diagnostic endovascular invasive device placed within the venous vasculature.

According to some embodiments, the designated substance/s is configured to aid in the detection of cell death.

According to some embodiments, the administration is designated to be optimized based on its therapeutic effect detected by the at least one sensor of the diagnostic endovascular invasive device.

According to some embodiments, the designated substance/s may be detected by an external sensor such as an ultrasound sensor or magnetic detection equipment.

According to some embodiments, the at least one sensor is a chemical sensor configured to detect inorganic or organic ions such as Potassium, Phosphates, Sodium, Calcium, Copper, Hydronium, Lactate and others.

According to some embodiments, the sensor is based on an Ion-Specific Field Effect Transistor (ISFET)/is an electrochemical cell with at least 2 electrodes made of at least one material/is configured to detect the ionic strength of the blood.

According to some embodiments, the at least one sensor is an optical sensor configured to detect cell residues characteristic of cell death such as membrane blebs, nucleic acids, DNA or RNA fragments, exosomes, nucleic fragments, structural proteins, mitochondrial proteins or other cellular debris, biochemical signals or others.

According to some embodiments, the sensor is of an optical type such as a UV-VIS spectrometer, NIR spectrometer, FTIR spectrometer or other/is configured to detect the second harmonic generation signature of Collagen.

According to some embodiments, the at least one sensor is a biological sensor configured to detect cell residues characteristic of cell death and/or is based on an immunosorbent assay/fluorescent markers.

According to a second aspect, there is provided a medical method, comprising the steps of (i) inserting at least one diagnostic endovascular invasive device that comprises at least one sensor into the venous vasculature, (ii) utilizing the at least one sensor in order to detect biochemical or physiological changes in a bodily fluid, (iii) analyzing the data captured by the at least one sensor using a controller, wherein the at least one diagnostic endovascular invasive device is designated to be in a proximity to a target tissue, and wherein the controller is designated to process received data in order to produce medical recommendations and/or conclusions.

According to some embodiments, the method further comprising a step of inserting a therapeutic endovascular invasive device into the arterial vasculature and administrate a designated substance/s in order to affect the at least one sensor of the diagnostic endovascular invasive device placed within the venous vasculature.

According to some embodiments, a new medical catheter based system is provided, comprising:
(i) at least one diagnostic endovascular invasive (EVI) device comprising at least one lumen, configured to be inserted into the venous vasculature, and positioned in proximity to a target tissue,
(ii) at least one sensor configured to be integrated with the at least one diagnostic endovascular invasive device and configured to monitor and detect biochemical and/or physiological changes in a venous fluid, at least partially originated from the target tissue, and
(iii) a controller configured to receive the monitored and detected data derived from the at least one sensor, to process and analyze the data and produce medical recommendations and/or conclusions.

According to some embodiments, at least one of the following holds true:
target tissue is a tumor and wherein the sensors/s are configured to monitor biochemical and/or physiological changes in a fluid stream at least partially originated from the tumor;
the device further comprises visual internal control means configured to provide spectroscopic control reference to the sensor/s;
at least two sensors are geometrically arranged in a staggered way, configured to deduce spatial and spatiotemporal information regarding biochemical and/or physiological signals, found in proximity to the device;
at least one sensor is an optical sensor configured to detect cell residues characteristic of cell death; cells are at least one of: membrane blebs, nucleic acids, DNA or RNA fragments, exosomes, nucleic fragments, structural proteins, mitochondrial proteins or other cellular debris, biochemical signals and any combination thereof;
at least one sensor is of an optical type selected from a UV-VIS spectrometer, NIR spectrometer, FTIR spectrometer, led, optical fiber and any combination thereof;
at least one sensor is configured to detect the second harmonic generation signature of Collagen.

According to some embodiments, the diagnostic endovascular invasive device comprises at least two lumens, wherein the first lumen comprises the at least one sensor and the second lumen comprises operational means.

According to some embodiments, the second lumen comprises at least one of:
a guide-wire, configured to be at least temporarily integrated within the second lumen, and to guide the device towards the target tissue; and
openings configured to draw fluid for external analysis, and/or to infuse a designated substance/s having an effect on the fluid surrounding the at least one sensor in the first lumen.

According to some embodiments, at least one of the flowing holds true:
the infused substance/s is designated to prevent blood clots in the proximity of the at least one sensor; and.
the infused substance/s is designated to improve the sensing capabilities of the sensor/s.

According to some embodiments, the system further comprising a therapeutic endovascular invasive device configured to be inserted into an arterial vasculature and administrate a designated substance/s configured for at least one of:
infuse an arterial inflow to the target tissue; and
affect the at least one sensor.

According to some embodiments, at least one of the following holds true:
the designated substance/s is configured to aid in the detection of cell death;
the administration is designated to be optimized based on its therapeutic effect detected by the sensor/s; and
the designated substance/s is configured be detected by an external sensor selected from: an ultrasound sensor and/or magnetic detection equipment.

According to some embodiments, the at least one sensor is a chemical sensor configured to detect inorganic and/or organic ions.

According to some embodiments, the sensor is at least one of:
based on an Ion-Specific Field Effect Transistor (ISFET);
an electrochemical cell with at least two electrodes made of at least one material;
configured to detect inorganic and/or organic ions selected from: Potassium, Phosphates, Sodium, Calcium, Copper, Hydronium, Lactate and any of their combination; and
configured to detect the ionic strength of the fluid.

According to some embodiments, the at least one sensor is a biological sensor configured to detect cell residues characteristic of cell death.

According to some embodiments, the sensor is at least one of:
based on an immunosorbent assay; and
based on fluorescent markers.

According to some embodiments, a new medical method is provided, comprising the steps of:
(i) inserting at least one diagnostic endovascular invasive device, comprising at least one lumen and at least one integrated sensor, into a venous vasculature, such that the sensor/s is/are placed at a proximity to a target tissue,
(ii) monitoring biochemical and/or physiological venous fluid at least partially originated from the target tissue, via the sensor/s, and detecting biochemical and/or physiological changes in the venous fluid, and
(iii) analyzing the monitored and detected data using a controller, for producing medical recommendations and/or conclusions.

According to some embodiments, the method further comprising a step of inserting another therapeutic endovascular invasive device into the arterial vasculature and administrating a designated substance/s configured for at least one of:
affecting the at least one sensor; and
infusing the target tissue.

According to some embodiments, the steps of monitoring, detecting and analyzing of the venous fluid are provided in real-time, while the target tissue is infused.

According to some embodiments, the method further comprising selecting the administrated substance/s, based on the real-time analysis, for optimizing a treatment for the target tissue.

According to some embodiments, the method further comprising detecting cell residues characteristic of cell death.

According to some embodiments, the step of inserting comprises placing the sensor/s at the proximity to the target tissue for preventing biochemical signals from being diluted and/or degraded in the fluid stream before the monitoring and detecting.

According to some embodiments, the device comprises at least two lumens and the method further comprising steps of:
integrating at least one sensor at the first lumen; and
at least one of:
   guiding the device towards the target tissue, via a guide-wire, at least temporarily integrated in the second lumen;
   drawing fluid for external analysis via the second lumen, and
   administrating a designated substance/s via the second lumen, configured for affecting the sensor/s at the first lumen.

According to some embodiments, the method further comprises at least one of:
preventing blood clots in the proximity of the sensor/s, via the designated substance/s;
improving the sensor/s sensing capabilities, via the designated substance/s.

According to some embodiments, the method comprising removing and/or replacing the integrated sensor/s via the first lumen.

According to some embodiments, the step of detecting comprises at least one of:
detecting an increase/decrease in ion concentration in a fluid stream;
detecting ionic strength of the fluid;
discerning between different materials in an analyte;
providing optical excitation and/or measuring reflection; and
detecting the second harmonic generation signature of Collagen.

According to some embodiments, a new medical system is provided, comprising:
one or more catheters each comprising at least one lumen, configured to be inserted, via an endovascular invasive (EVI) device, into one or more venous vasculatures, and positioned in proximity to one or more target tissues;
one or more sensors configured to be integrated with at least one of the catheters distal-end, and configured to monitor and/or detect biochemical-and/or physiological-changes in a venous fluid, at least partially originated from the target tissue/s, and
an implant port, configured to be connected to the proximal-end of the catheter/s, for communication therebetween; the port comprising:
   one or more reservoirs, each having an access-element, and each configured to be in fluid communication with at least one of the catheters;
   a data-storage module, configured to communicate with the sensor/s and to store the monitored-and/or detected-data derived therefrom; and
   a wireless receiver-and/or transmitter-module, configured for wireless communication between the data-storage module and a controller.

According to some embodiments, the access-element is configured to enable exterior fluid communication with its associated reservoir and therefore with the reservoir's associated catheter/s, for at least one of:
flushing the associated catheter/s'-lumen/s and/or-sensor/s;
providing a therapeutic and/or diagnostic infusion, via the associated catheter/s; and
obtaining aspiration sample/s, via the associated catheter/s.

According to some embodiments, the port further comprises one or more pumps, configured to draw and/or push fluid between the reservoir/s and it's/their associated catheter/s.

According to some embodiments, the pump is configured to control the fluid communication between the reservoir and it's/their associated catheter/s.

According to some embodiments, the port comprises a single said reservoir, configured to be connected with- and in fluid communication with- at least two of the catheters, and wherein:
a first said catheter is configured to be positioned in proximity to the target tissue; and
a second said catheter is configured to be positioned in proximity to a right atrium; such that an influence of a treatment provided to the target tissue, via the second catheter, is monitored and/or detected via the first catheter.

According to some embodiments, each of the catheters comprises at least one blocking-element, configured block its associated lumen/s, thereby configured to enable at least one of:
blocking the lumen/s of the first catheter, when pushing and/or drawing fluid via the second catheter;
blocking the lumen/s of the second catheter, when pushing and/or drawing fluid via the first catheter; and
blocking the lumen/s of both the first-and second-catheters, to enable cleansing of the reservoir.

According to some embodiments, each of said reservoir/s is configured to be connected with- and in fluid communication with- only one of said catheters.

According to some embodiments, the port comprises at least two said reservoirs, and wherein:
a first said reservoir is configured to be connected to a first said catheter, configured to be positioned in proximity to the target tissue; and
a second said reservoir is configured to be connected to a second said catheter, configured to be positioned in proximity to a right atrium;
   such that an influence of a treatment provided to the target tissue, via the second catheter, is monitored and/or detected via the first catheter.

According to some embodiments, each of the access-elements is differently marked, configured to enable a caregiver an identification of its associated catheter.

According to some embodiments, wherein the implant port is a subcutaneous port, configured to be implanted under skin.

According to some embodiments, the access-element comprises a septum, configured to enable fluid to be injected to-and/or to be drawn from-the reservoir, via a needle.

According to some embodiments, the port further comprises at least one power source. According to some embodiments, at least one of the power sources is a wireless rechargeable battery.

According to some embodiments, the port comprises a controller module, configured communicate via the receiver-and/or transmitter-module with other controllers.

According to some embodiments, the controller module is configured to receive the monitored and/or detected data derived from the at least one sensor, to process and analyze their data and produce medical recommendations and/or conclusions.

According to some embodiments, at least one of the following holds true:
the target tissue is a tumor and wherein the sensors/s are configured to monitor biochemical and/or physiological changes in a fluid stream at least partially originated from the tumor;
the controller further comprises visual internal control means, configured to provide spectroscopic control reference to the sensor/s;
at least two sensors are geometrically arranged in a staggered way, configured to deduce spatial and spatiotemporal information regarding biochemical and/or physiological signals, found in proximity to the device;
at least one sensor is an optical sensor configured to detect cell residues characteristic of cell death; cells are at least one of: membrane blebs, nucleic acids, DNA or RNA fragments, exosomes, nucleic fragments, structural proteins, mitochondrial proteins or other cellular debris, biochemical signals and any combination thereof;
at least one sensor is of an optical type selected from a UV-VIS spectrometer, NIR spectrometer, FTIR spectrometer, led, optical fiber and any combination thereof;
at least one sensor is configured to detect the second harmonic generation signature of Collagen;
at least one sensor is a chemical sensor configured to detect inorganic and/or organic ions; and
at least one sensor is a biological sensor configured to detect cell residues characteristic of cell death.

According to some embodiments, a new medical method is provided, comprising the steps of:
inserting at least one diagnostic-catheter, via an endovascular invasive (EVI) device, comprising at least one lumen and at least one integrated sensor, into a venous vasculature, such that the sensor/s is/are placed at a proximity to a target tissue;
implanting a subcutaneous port, comprising
   one or more reservoirs, each reservoir having an access-element;
   a data-storage module, configured for communicating with the sensor/s and to storing the monitored-and/or detected-data derived therefrom;
   a wireless receiver-and/or transmitter-module, configured for wireless communication between the data-storage module and a controller;
connecting the reservoir/s to the proximal-end of the inserted diagnostic-catheter/s for communication therebetween; each reservoir to a different catheter;
monitoring biochemical and/or physiological venous fluid at least partially originated from the target tissue, via the sensor/s, and detecting biochemical and/or physiological changes in the venous fluid, and
analyzing the monitored and/or the detected data using the controller, for producing medical recommendations and/or conclusions.

According to some embodiments, the method further comprises at least one of:
drawing fluid for external analysis, via diagnostic-catheter and a first reservoir;
infusing designated substance/s having an effect on the fluid surrounding the at least one sensor, via diagnostic-catheter and a first reservoir; and
removing and/or replacing sensor/s integrated on diagnostic-catheter, via an EVI device.

According to some embodiments, the method further comprises at least one of:
inserting a therapeutic-catheter, via an EVI device, such that its distal-end is in proximity to a right atrium, and connecting a second- and different-reservoir of the subcutaneous port to a proximal-end of the inserted therapeutic-catheter, for communication therebetween; and.
infusing the right atrium with a systematic therapeutic treatment via the therapeutic-catheter and the second reservoir, and monitoring and/or detecting an influence of the provided therapeutic treatment for a target tissue, via the diagnostic catheter.

According to some embodiments, the steps of monitoring, detecting and analyzing of the venous fluid are provided in real-time, while the target tissue is infused.

### BRIEF DESCRIPTION OF THE FIGURES

Some embodiments of the invention are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the invention.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 constitutes a schematic perspective view of a typical needle biopsy of a solid tumor;

FIG. 2 constitutes a schematic perspective view of a tumor located in the liver;

FIG. 3 constitutes a schematic perspective view of a typical vascular flow through a tissue;

FIG. 4 constitutes a schematic perspective view of a diagnostic and therapeutic system being in conjunction to an abnormal mass, according to some embodiments of the invention;

FIG. 5 constitutes a schematic perspective view of the tip of a catheter based diagnostic device being in conjunction to an abnormal mass, according to some embodiments of the invention;

FIG. 6 constitutes a schematic perspective view of a proximal catheter hubs/connectors forming a part of diagnostic and therapeutic system, according to some embodiments of the invention;

FIG. 7A constitutes a schematic perspective view of a relationship between the distal to more proximal sensor tip of a double tip catheter based diagnostic device that forms a part of diagnostic and therapeutic system, according to some embodiments of the invention;

FIG. 7B constitutes a schematic perspective view of a compact structure of catheter based diagnostic device having a telescopic lumen structure, according to some embodiments of the invention;

FIG. 7C constitutes a schematic perspective view of an optical internal control forming a part of catheter based diagnostic device, according to some embodiments of the invention;

FIG. 8A-8B constitute a schematic perspective view of a side-by-side double lumen and “telescopic” single lumen forming a part of catheter based diagnostic device, according to some embodiments of the invention;

FIG. 9A-9C constitute a schematic perspective view of the position and functionality of staggered side sensors and tip sensor embedded in catheter based diagnostic device forming a part of diagnostic and therapeutic system, according to some embodiments of the invention;

FIG. 10A graphically illustrates results of variable levels of phosphate ion concentration in a physiological fluid, according to some embodiments of the invention.

FIGS. 10B-10C graphically illustrate experimental results of phosphate concentration measurement using gold working electrode and platinum counter electrode, and experimental results of phosphate concentration measurement using gold working electrode and platinum counter electrode, respectively and according to some embodiments of the invention;

FIG. 11 graphically illustrates a side cut view of a catheter-based medical device comprising a port, according to some embodiments of the invention;

FIG. 12A graphically illustrates another side cut view of a catheter-based medical device comprising a port, according to some embodiments of the invention;

FIG. 12B graphically illustrates a cut view of a diagnostic catheter, having a single lumen and exterior wall layers, configured to accommodate sensors and/or wires, according to some embodiments of the invention;

FIG. 13A graphically illustrates a top view of a catheter-based medical device having a single reservoir connected to two catheters (diagnostic and therapeutic), according to some embodiments of the invention;

FIG. 13B graphically illustrates a top view of a catheter-based medical device having two reservoirs, each connected to a different catheter (diagnostic and therapeutic), according to some embodiments of the invention; and

FIG. 14 graphically illustrates a method of using a catheter-based medical device, demonstrating the implanted port connection with a diagnostic catheter leading to the liver and a therapeutic catheter leading to the right atrium, according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components, modules, units and/or circuits have not been described in detail so as not to obscure the invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “controlling” “processing,” “computing,” “calculating,” “determining,” “establishing”, “analyzing”, “checking”, “setting”, “receiving”, or the like, may refer to operation(s) and/or process(es) of a controller, a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Unless explicitly stated, the method embodiments described herein are not constrained to a particular order or sequence. Additionally, some of the described method embodiments or elements thereof can occur or be performed simultaneously, at the same point in time, or concurrently.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the present disclosure.

Throughout this application, various embodiments described may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Various embodiments and aspects of the present disclosure as delineated hereinabove and as claimed in the claims section below find experimental support in the following examples.

The terms “comprises”, “comprising”, “includes”, “including”, “having” and their conjugates mean “including but not limited to”.

As used herein, the singular form “a”, “an” and “the” include plural references unless the context clearly dictates otherwise. For example, the term “a compound” or “at least one compound” may include a plurality of compounds, including mixtures thereof.

As used herein, the term “about” refers according to some embodiments, to a range selected from: ±15%, ±10%, ±5%, ±1%, and any combination thereof.

The term “port” as used herein, refers according to some embodiments, to an implanted port, often called a mediport or port-a-cath. According to some embodiments, the parts of the implanted port comprise a fluid reservoir and an access element, which can also be referred as an access point (e.g., septum). According to some embodiments, the port is configured to be connected with a catheter, where the reservoir is starting or ending point for the fluids flow through the catheter, depending on the method of use. According to some embodiments, the port is configured to be implanted under the skin and comprises a raised center access point (e.g., the septum). According to some embodiments, the access point is configured to enable the insertion of a needle, or a similar fluid delivering element, without contaminating the reservoir.

According to some embodiments, the port and its connected catheter are configured to deliver into the bloodstream at least one of: medicine, chemotherapy, intravenous fluids, blood transfusions, blood products, nutrients, and fluids. The terms “systematic therapy” and “systematic treatment” as used herein, refer according to some embodiments, to fluidic therapies provided via the right atrium and adapted to work-throughout the whole of the body.

According to some embodiments, septum is made from a self-sealing rubber material or from similar characteristic materials. According to some embodiments, fluids cannot be inserted to the port, without a needle use. According to some embodiments, the septum is configured to be closed, once the needle is removed.

According to some embodiments, the port's catheter is a thin, flexible tube, made from medical grade materials serving a broad range of functions. According to some embodiments, a catheter is a medical device that can be inserted into the body to treat diseases and/or perform a surgical procedure. According to some embodiments, catheters are manufactured and are configured for a use in applications, such as cardiovascular, urological, gastrointestinal, neurovascular and ophthalmic procedures and more.

According to some embodiments, one end (the proximal end) of the catheter is configured to be connected to the port. According to some embodiments, the other end (the distal end) is configured to be placed in a vein, an artery and/or any tissue lumen, according to some embodiments, in proximity to a target tissue. According to some embodiments, the catheter is configured to be inserted into a vein and/or artery via the use of an endovascular invasive (EVI) device.

The term “hub” as used herein, refers according to some embodiments, to a communication connecting element, for a non-limiting example, a central device that connects multiple electronic/computational elements, such as: memory/storage module, battery/s, electric cables, processors, controllers, sensors, receiver- and/or transmitter-module and the like.

The term “treatment”, as used herein, refers to a therapeutic intervention that, for example, ameliorates a sign or symptom of a disease or a pathological condition, inhibits the progression of a disease or a pathological condition, delays worsening of a disease or a pathological condition, and even completely prevents the development of a disease or a pathological condition. The term “treatment protocol”, as used herein, refers to a precise and detailed plan for managing and/or treating a disease or condition.

The term “Administration”, as used herein, is provision or introduction into a subject, by a chosen route, of one or more active agents (e.g. drugs) for therapeutic or diagnostic purposes. Systemic administration is a route of administration of medication, nutrition and/or other substance into the circulatory system so that the entire body is affected. Systemic administration can take place via enteral administration (absorption of the drug through the gastrointestinal tract) or parenteral administration (e.g., injection, infusion, or implantation). Systemic routes of administration include, for example, oral, nasal, rectal, dermal, intravenous, intracardiac, intramuscular, intraperitoneal, intranasal, buccal, sublingual and/or topical administration. Local administration is delivering one or more active agents within, or directly, or almost directly to the site of action, which may be, for example, a lesion, tumor, organ, a transplant and/or the like. In local drug administration, the risk of systemic side effects is reduced. Local administration is exemplified by topical administration and/or in-situ injection or infusion.

The term “controller”, as used herein, refers to any type of computing platform or component that may be provisioned with a Central Processing Unit (CPU) or microprocessors, and may be provisioned with several input/output (I/O) electric ports, for example, a general-purpose computer such as a personal computer, laptop, tablet, mobile cellular phone, controller chip, SoC or a cloud computing system.

Many tumors are diagnosed at a stage when they are not amenable to surgical resection. In the cases, systemic treatment is commonly used. Systemic treatment is a drug either ingested or administered intravenously. In some circumstances, the drug can be delivered via the arterial supply of a tumor, such as in chemo-embolization or selective arterial infusion.

According to some embodiments, a diagnostic catheter is provided and designated to provide minimally invasive means of placing a plurality of sensors or mechanisms in a selective vein. According to some embodiments, the diagnostic catheter may be any known means of accessing vasculature such as needles, implants, ports, etc.

According to some embodiments, the invention comprises a system and methods configured to monitor the response of a target tissue that is being subjected to treatment. The tissue physiological and chemical response are monitored through a diagnostic device, which may be a minimally invasive catheter and the information collected is typically transferred to a controlling unit, in which quantitative analyses are being performed and information and conclusions are configured to be presented to a medical staff.

According to some embodiments, the use of a local diagnostic catheter allows to overcome previous limitations in other known sensing techniques, since placing it in a vein draining the target tissue prevents biochemical signals from being diluted or degraded in the blood stream before being detected.

According to some embodiments, placing a sensor-enabled catheter in the vein that exits the target tissue allows detecting relevant signals at the time and location most relevant and with the highest signal-to-noise potential ratios. Furthermore, the invention herein may be used in conjunction with the use of selective arterial infusion, whereby an arterial catheter is positioned in the arterial inflow to the tumor/target tissue.

According to some embodiments, the combination of selective arterial infusion and the heretofore described venous sensor-enabled catheter may be employed to assess the effects of infused therapeutics upon the target tissue in near real-time. Consequently, the choice of therapeutics, tissue-modulating agents and other infusible compounds is made based upon dynamic and observed results. According to some embodiments, the claimed invention can be used to obtain observed/empiric drug-specific diagnostic information and, by optimizing the infused therapy based on feedback, the claimed invention can be used not just for diagnostic purposes, but rather with therapeutic intent.

According to some embodiments, the claimed invention includes at least one sensor integrated on the diagnostic catheter and configured to either collect data, transmit data to a controlling unit or perform analyses in-situ. The data may involve biochemical signals of types described herein. According to some embodiments, potential chemical, biochemical, biological or physiological information may also be obtained by a diagnostic catheter.

According to some embodiments, the diagnostic catheter includes at least one chemical sensor that is configured to detect an increase in ion concentration in the blood stream. It is well established that in many tumors, a detectable increase in ionic concentration is present through most or all of the tumor's growth.

For example, the increase or decrease in ions, such as sodium, potassium, chloride, etc., as well as pH and lactate, from within a tumor draining vein are indicative of changing physiology, and often of cell death.

According to some embodiments, the detection of one or more ion species can be performed by integrated sensors on the diagnostic catheter. For example, Ion-Specific Field Effect Transistors (ISFETs) can be integrated on the catheter in such configuration to detect any of the abovementioned ions or others. Ion species such as ISFETs with SiO2 or SiN3 ion-sensitive membranes can be used to detect changes in pH, Hafnium Oxide membranes to detect changes in Potassium or Sodium ion concentrations, etc.

According to some embodiments, multiple ISFETs may be integrated on a single catheter for the purpose of multiple ion sensing and the ability to correct for interference between measurements such as a single sensing membrane electrically responding to more than one ionic concentration. Furthermore, in some embodiments, multiple similar ISFETs are placed on the catheter at different locations or orientations to allow analyses of the time-domain response such as the advective velocity in the blood stream.

According to some embodiments, the sensors' signals can be electrically analyzed at the tip of the catheter, multiplexed at the tip or be drawn galvanically to the external (proximal) part of the catheter and electrically treated outside of the patient's body. According to some embodiments, a use of Extended Gate Ion Specific Field Effect Transistor (EGISFET) is utilized to transfer the ionic signals on a sensitive film through a galvanic connection in the catheter to an external field effect transistor in a farther, more proximal location such as an external device. In yet other embodiment, the use of ISFETs can be enhanced for better sensitivity by using Dual-Gate ISFET (DGISFET).

According to some embodiments, in addition to or instead of ion or material sensing, the diagnostic catheter is integrated with sensors to detect general characteristic of the blood as an analyte. For example, the ionic strength of the liquid can be measured with the integration of such sensors as Crystalline Colloidal Arrays (CCA sensors) that typically respond to the ionic strength rather than to a specific ion species. Such sensors can be used to correct or bias other measurements alongside with corrections obtained by internal or external temperature sensing and flow velocity sensing that can be integrated in the system for calibration, correction or validation.

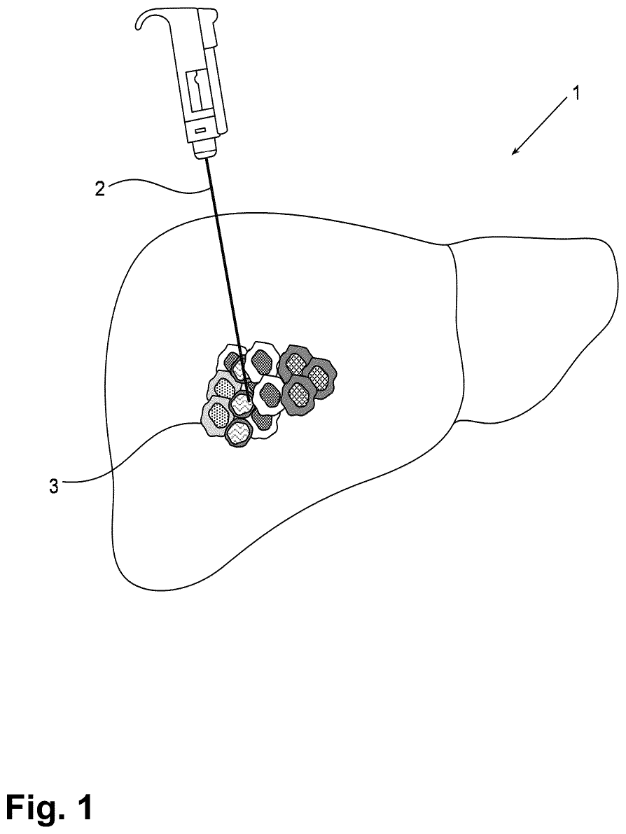

Reference is now made to FIG. 1 which schematically illustrates a representation of a typical needle biopsy of a solid tumor 3 within an organ 1. As shown, tumor 3 is composed of heterogeneous cell composition undergoes a biopsy using a needle 2. According to some embodiments, needle 2 has an inner diameter of 0.8 to 1.0 mm, as a result, even with several biopsies of a single tumor, there is ample opportunity for misrepresentation or in accurate representation of the various cells within a tumor.

Tumor heterogeneity can exist in several important respects: Cells within a single tumor can differ from each other in terms of their particular genetic mutations. Cancer cells accumulate mutations in their DNA over time, which can cause different cells within the tumor to have different mutations. As tumor cells multiply, the result can be heterogeneous patches of tumor subtypes within one mass. In addition to genetic differences, epigenetic variation such as DNA methylation differences can also be seen within a tumor (e.g, Glioblasgoma).

Within a single, solid tumor, further heterogeneity may be observed with regard to the tumor microenvironment, including such variations as the type of interstitial cells (fibroblasts, for example), immune cells (macrophages and lymphocytes, for example) and blood vessels in the immediate vicinity of the tumor cells. This heterogeneity may result in areas of increased, or decreased pH, oxygenation, necrosis, and interstitial pressure within a tumor.

Such heterogeneity may contribute to differences in how parts of a tumor respond to treatment. In this dynamic scenario of tumor treatment, a given therapy may also induce or accelerate the pace of change in sub populations of the treated tumor. Systemic therapy may result in the activation of adaptive feedback mechanisms (eg BRAF/EGFR). Systemic treatment may also change the tumor in more direct ways: tumor cells with mutations in a treatment-targeted gene may evolve to become more dominant. In addition, tumor cells may adapt by increasing expression of tumor efflux pumps.

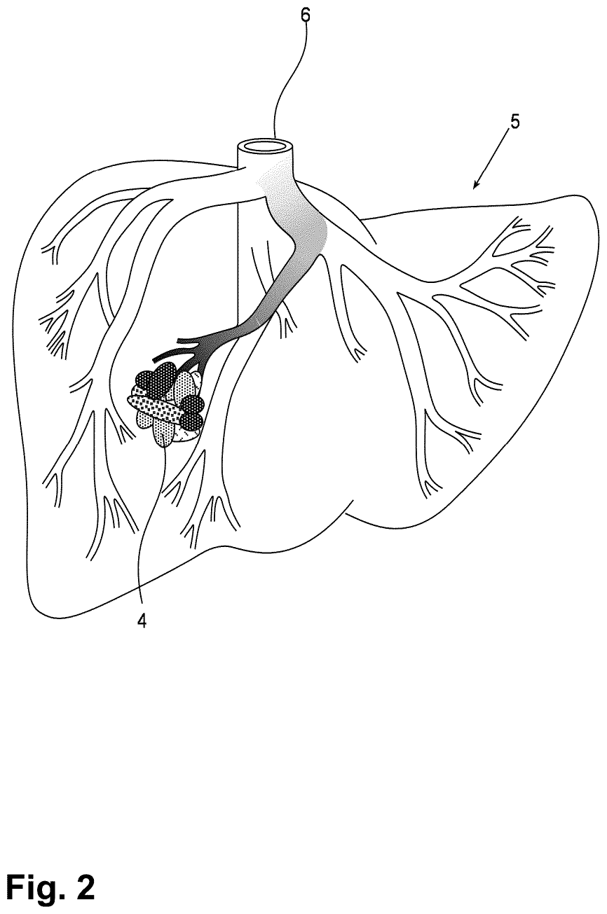

Reference is now made to FIG. 2 which schematically illustrates a tumor 4 located in the liver 5, with its venous blood being drained through an inferior branch of the middle hepatic artery 6. As shown, tumor specific venous blood is marked by a gradient of color, which is progressively diluted by the blood stream marked in dashed line, which is draining the normal non-tumor regions of the liver. For example, the tumor specific venous blood in close proximity to the tumor is in concentration of 100%, and after a certain distance is in concentration of 50%, etc.

It is important to note that the pathologic depiction in FIG. 2 may occur in any other organ or tissue. The venous gradient of tumor-specific products is a feature which can be leveraged in order to localize tumors, which are too small, or otherwise difficult to find using conventional medical imaging.

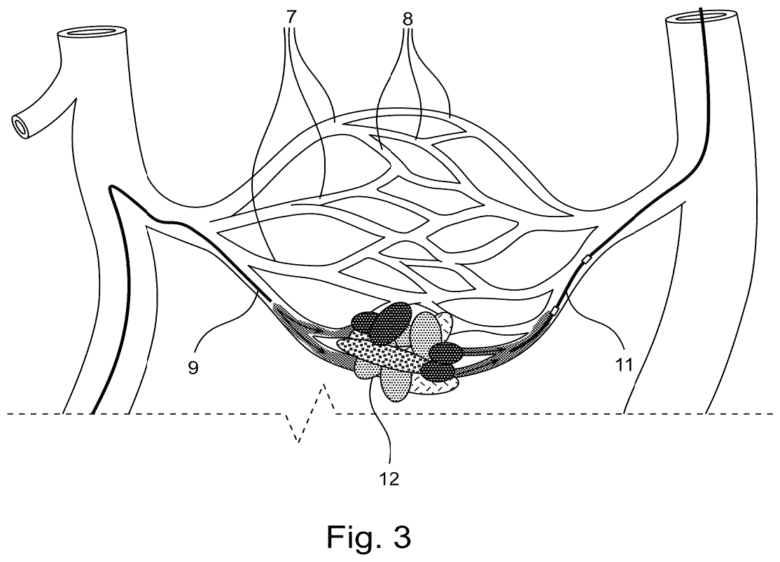

Reference is now made to FIG. 3 which schematically illustrates a vascular flow through tissue. As shown, branching arteries 7, divide into smaller arterials and capillaries 8 and accumulate into branching veins which combine to form larger veins at each confluence.

According to some embodiments, the selective arterial supply 9 to a tumor 12 and its venous drainage 11 is seen in the lower aspect of the tissue. According to some embodiments, a diagnostic catheter of the claimed invention is designated to be positioned in the selective tumor venous outflow and an arterial catheter/micro-catheter is designated to be positioned in the selective arterial supply to the tumor as further disclosed below. It should be noted that for the purpose of achieving local therapeutic delivery, a percutaneous or otherwise placed needle with access to the target tissue would be equivalent to arterial access or any known accesses technique.

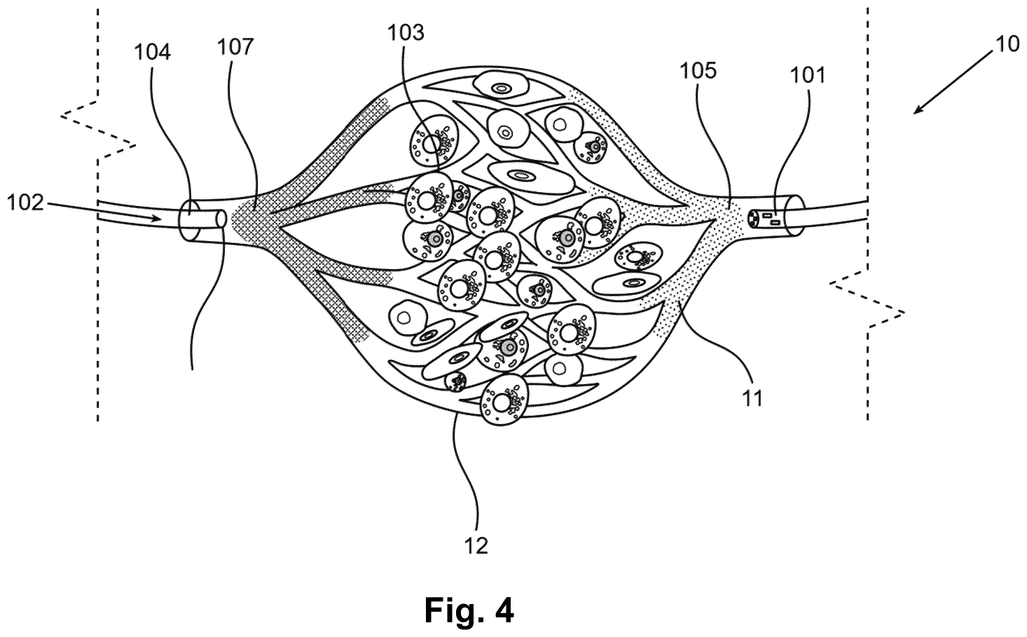

Reference is now made to FIG. 4 which schematically illustrates a diagnostic and therapeutic system 10 being in conjunction to an abnormal mass 103. As shown, an abnormal mass 103 in a patient's body as is typically presented in physiological schemes. The abnormal mass 103 typically draws vasculature to sustain its growth, and has at least one artery 107 leading to it and at least one vein 105 leading out of it.

According to some embodiments, the vasculature disclosed above may serve a care-giver as access to the vicinity of the abnormal mass 103 for treatment, and specifically for the introduction of a catheter based diagnostic device 101 for the introduction of therapeutic payloads of different types. According to some embodiments, the arterial catheter 102 may have at least one lumen designated for the flow of at least one active ingredient such as a chemotherapeutical payload, etc. According to some embodiments, a catheter based diagnostic device 101 may have at least one lumen designated to accept and analyze at least one diagnosable substance secreted or being discharged from the abnormal mass 103. According to some embodiments, the arterial catheter 102 may be designated to be inserted into artery 107 that supplies blood to abnormal mass 103 and the catheter based diagnostic device 101 may be designated to be positioned into a vein 105 draining the abnormal mass 103.

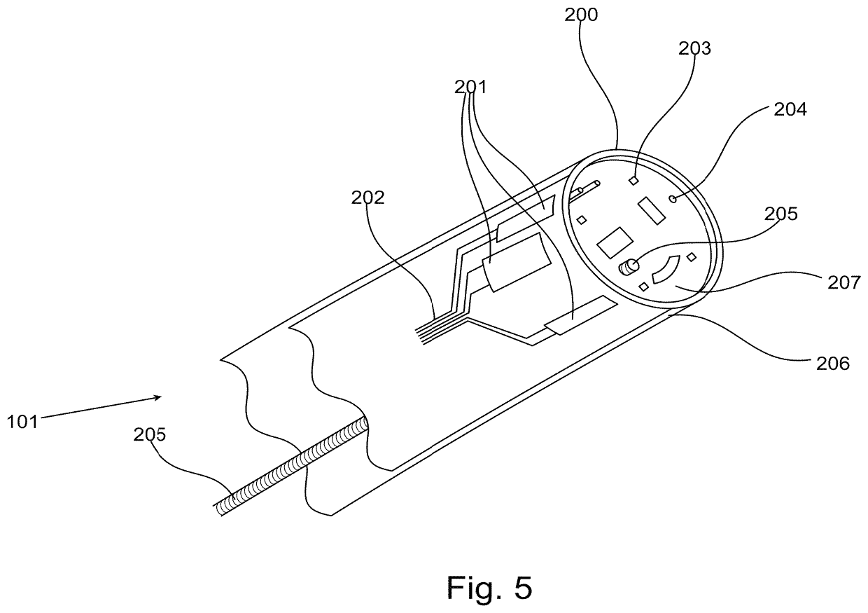

Reference is now made to FIG. 5 which schematically illustrates the tip of the catheter based diagnostic device 101 being in conjunction to an abnormal mass 103 (not shown). As shown, catheter tip 200 comprises multiple ISFET sensors 201, 207 integrated at different locations and allows multiple substances detection. According to some embodiments, FET based sensors are disclosed as a general type of sensors used to represent some of the multiple embodiments described herein.

According to some embodiments, at least one sensor/s 201 may be configured and placed on wall 206 in a way that exposed it to the venous blood stream for interaction with outflowing bio-chemical substances. For example, sensor/s 201 may be located on the sides of the catheter, on its tip, etc.

According to some embodiments, sensor/s 201 may be controlled and read by connectors 202 that may be galvanic connections, through which the voltages and currents are supplied or measured respectively. Further to the direct chemical sensing, spectrometry related sensing are represented as potentially forward looking such as optical fibers 203 that can provide an excitation and reflection measurements, and potentially and utilize other light sources such as local LEDs 204.

According to some embodiments, catheter based diagnostic device 101 comprises mechanical components such as guidewire-accommodating element 205 configured to provide mechanical steering and connectivity.

According to some embodiments, sensor/s 201 may be at least one electrochemical sensor, meaning at least two electrodes with some voltage applied between them and where the current is measured, or vice-versa, current applied and voltage measured. The information collected from the relation between voltage and current may be referred to as Electrochemical Impedance Spectroscopy (EIS) in the genera case, and voltammetry in a private case of low frequency scanning, and has the potential to discern between different materials in an analyte.

According to some embodiments, at least two inert and bio-compatible electrodes may be made of one or more materials such as Platinum, Gold, Palladium, Titanium or some biocompatible alloys such as Magnesium alloys, stainless steel, etc. may be incorporated as sensors. An electrical reaction may occur between the electrodes and on the electrode surface. The collected data can be used to detect and quantify the abovementioned characteristic inorganic or organic ions.

According to some embodiments, one example of using the structure is the use of two Platinum electrodes, one as a working electrode (WE) and one is a counter electrode (CE), and performing a linear sweep of voltage while measuring the resulting current to deduce when reduction or oxidation reactions occurred. Such information is pertinent to the ionic species present in the blood stream and to their quantification.

Reference is now made to FIG. 6 which schematically illustrate a proximal catheter hubs/connectors forming a part of diagnostic and therapeutic system 10. As shown, the proximal section of the catheter may include a designated guide wire 302 & infusion lumen/port 304 with a Luer lock compatible hub 306.

According to some embodiments, the interface between the various sensor fibers and light source embedded within catheter based diagnostic device 101 are optionally bundled into one or more hubs 306 for connection to a controller (not shown) such a computing consul designated to process and analyse data received from the catheter based diagnostic device 101 and provide an energy and light source.

According to some embodiments, catheter based diagnostic device 101 is configured to be connected to the controller by a wired and/or a wireless connection in order to enable a data transfer and analysis.

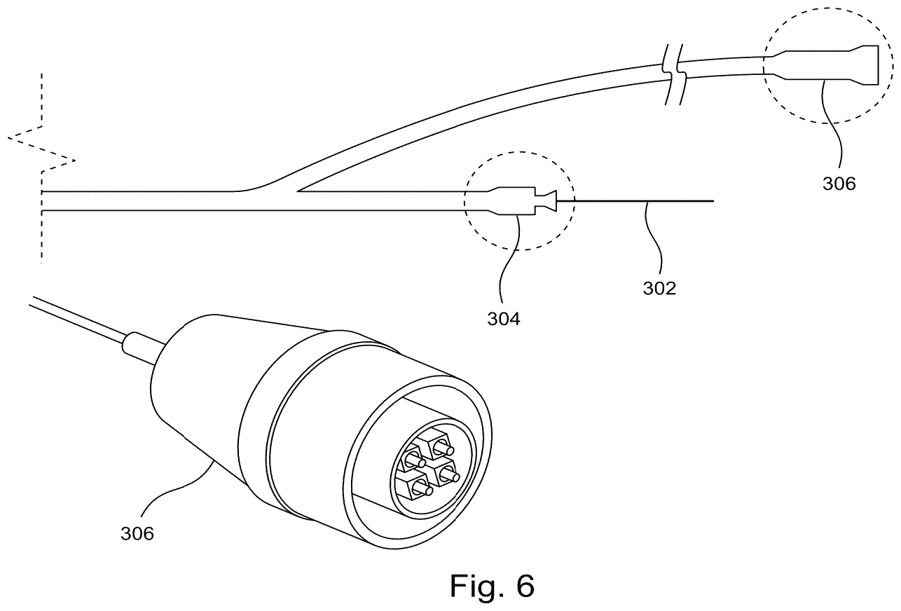

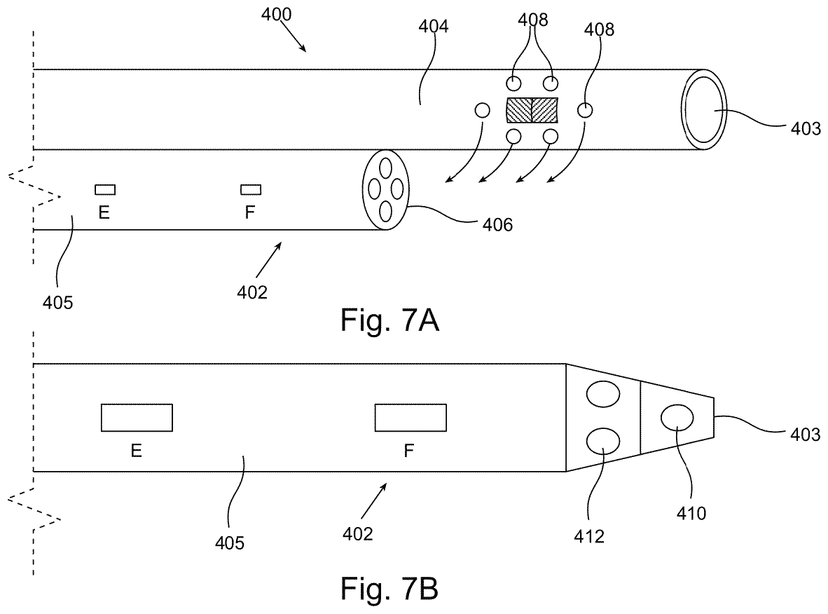

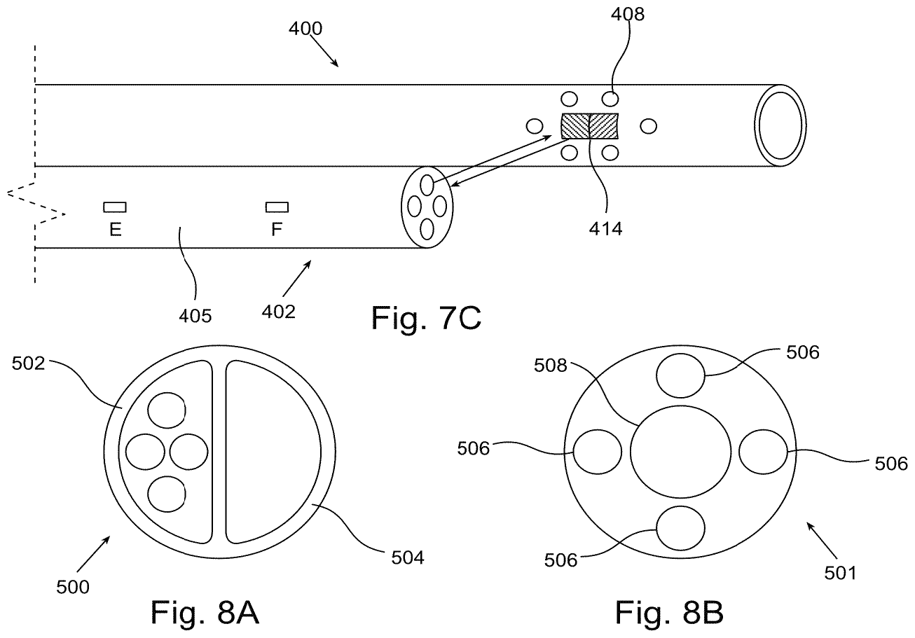

Reference is now made to FIGS. 7A, which schematically illustrates the relationship between the distal to more proximal sensor tip of a double tip catheter based diagnostic device 101 that forms a part of diagnostic and therapeutic system 10. As shown, the distal tip 403 of catheter based diagnostic device 101 has a dual lumen structure comprising lumens 400 and 402 or a telescopic lumen structure further disclosed below. According to some embodiments, the proximal catheter lumen 402 is occupied by sensors appropriate for assessing the composition of blood in vivo, as further described herein and the distal catheter

According to some embodiments, the distal tip of lumen 400, is configured to allow for the use of guidewires during endo-venous navigation. Once the catheter based diagnostic device 101 has arrived in location for venous sensing, the guide wire (not shown) may be removed. At this point, the distal catheter tip 403 is designated to serve as a point of fluid infusion and/or blood aspiration through designated openings 408 located on wall 404 as disclosed below.

According to some embodiments, on the wall 405 between the distal lumen and the proximal sensing tip 406 there is at least one sensing side hole E and/or F positioned such that the infuscate is directed to the space between the incoming venous blood and the sensors 412 at the proximal catheter tip 406.

According to some embodiments, such configuration enables a constant maintenance and inspection of the sensors found in proximal catheter lumen 402 by, for example, providing a constant flow of blood thinners from openings 408 in order to prevent blood clots from forming on proximal catheter tip 406 and sensing side hole E and/or F. In another example, the constant infusion of a certain substance from openings 408, may provide a control reference to the sensitivity of the various sensors found in catheter lumen 402.

Reference is now made to FIGS. 7B, which schematically illustrates a compact structure of catheter based diagnostic device 101 having lumen 402 a telescopic lumen structure further disclosed below. As shown, various sensors 412 are incorporated along the circumference of the catheter based diagnostic device 101 and terminate proximally to the distal lumen. In this configuration the at least one side holes remain in the distal lumen between distal tip and the proximal sensors.

According to some embodiments, distal infusion from the end hole 403 and side holes 410 may serve to promote and maintain optimal conditions for catheter based diagnostic device 101's operations. For example, a low-dose infusion of anticoagulants or other pharmacologic may serve to inhibit protein adherence, and/or clot formation around the indwelling sensors embedded within the catheter lumen 402.

According to some embodiments, the end hole 403 and side holes 410 may also be used to infuse reagents into the draining venous blood in order to optimize detection of specific molecules/substances of interest. For example, fluorescent probes or other reagents may be infused locally to interact with and identify specific molecules. Similarly, the distal infusion openings 408 (depicted in FIG. 7A) may serve to optimize local conditions for detection, including such changes as temperature and pH.

According to some embodiments, the catheter end hole 403 and side holes 410 can be used for operational procedures such as aspirating venous blood, which can then be subjected to further laboratory analyses such as advanced genomics and proteomics.

Reference is now made to FIGS. 7C, which schematically illustrates an optical internal control forming a part of catheter based diagnostic device 101. As shown, catheter based diagnostic device 101 may include internal sensor controls 414. For example, (especially if spectroscopic control may be positioned on the external circumference of the catheter lumen 402), in close proximity to the optical sensors may be installed. Another example of such an internal control would be composed of material of the same spectroscopic signature as in a molecule of interest for example, free DNA.

According to some embodiments, internal controls are configured to ensure accuracy and reliability of the sensors and potentially alert the operator of technical malfunction or other contamination.

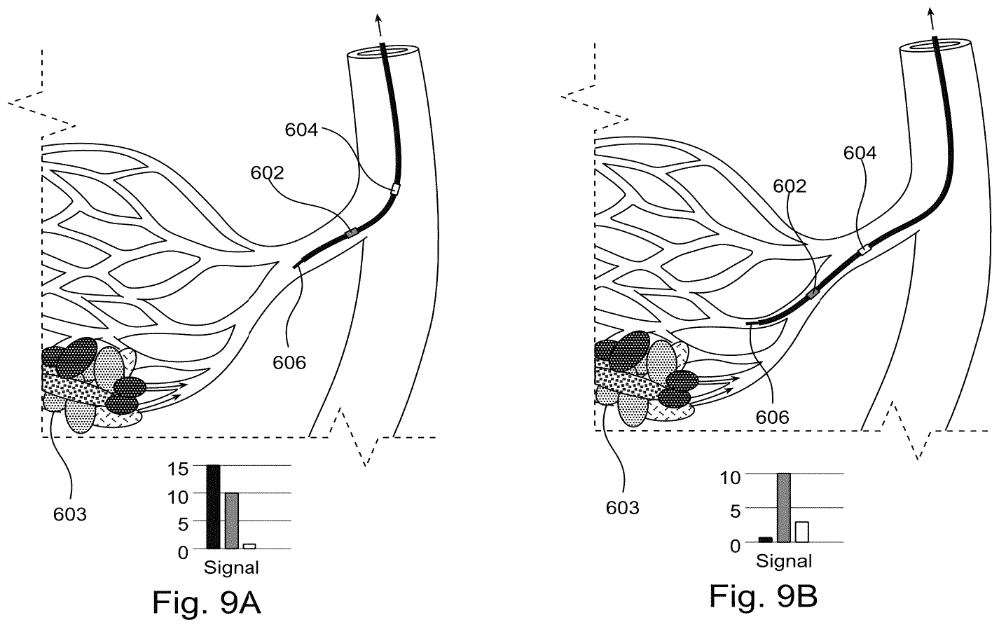

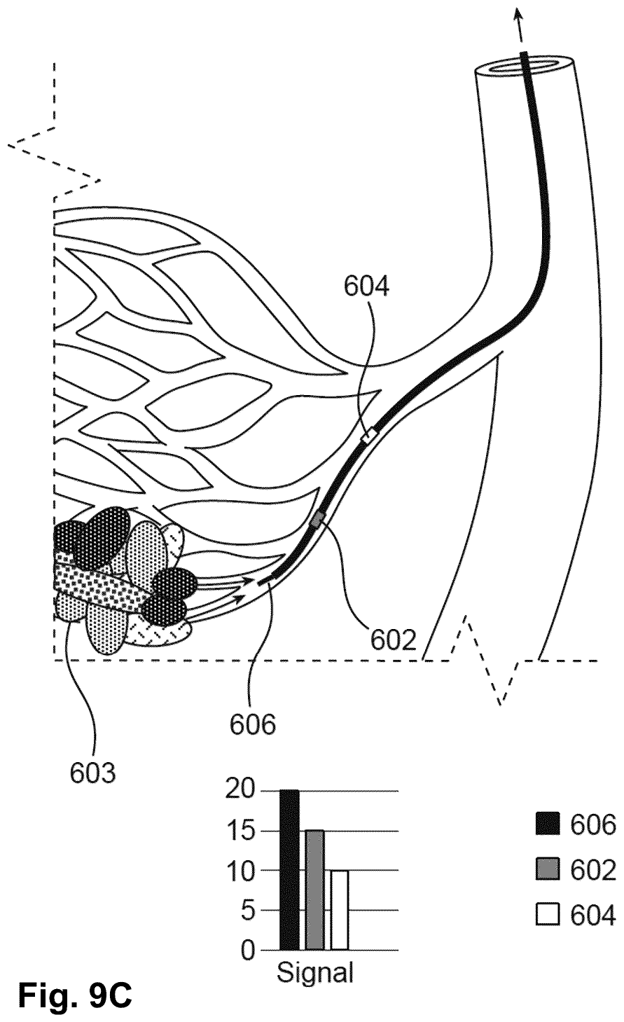

Reference is now made to FIGS. 8A and 8B, which schematically illustrate side-by-side double lumen and “telescopic” single lumen forming a part of catheter based diagnostic device 101. As shown, FIG. 9A depicts the side-by-side dual lumen structure catheter based diagnostic device 101.

According to some embodiments, catheter lumen 504 may accommodate a guide wire or vascular snare for endovascular navigation (not shown) and optionally a micro-drawing/infusion channel configured to draw blood for periodical external analyses, or for infusion, as described below. The other lumen 502 is occupied by a combination of sensors 506. According to some embodiments, dual lumen 500 that comprises the sensor lumen 502 may be exchanged for a different configuration of sensors or temporary early removed and replaced if maintenance of the sensors is required. Optionally, other embodiments will include additional sensor lumens' configuration.

According to some embodiments and as depicted in FIG. 8B, sensors 506 are incorporated into a telescopic lumen structure 501, having a peripheric/tubular structure forming a part of the catheter based diagnostic device 101. The configuration comprises a single through lumen 508 centrally extending through and reaching distal to the position of the sensors 506.

Reference is now made to FIGS. 9A-9C, which schematically illustrate position and functionality of staggered side sensors 602, 604 and tip sensor 606 embedded in catheter based diagnostic device 101 forming a part of diagnostic and therapeutic system 10.

According to some embodiments, and as disclosed above, during venous navigation of catheter based diagnostic device 101, there is a detectable, signal gradient originating within the target tumor, which is caused by a gradient of substances secreted from the tumor. The signal is diluted at every venous confluence. As seen in FIGS. 4A-4C, a progressive increase in signal is indicative of progression toward the source (e.g. a tumor).

According to some embodiments, in the event of a wrong turn which direct the catheter tip away from tumor 603, sensor 606 at the catheter tip will register a lesser signal relative to the side sensor 602 (as can be seen in FIG. 4B). In this instance, the catheter can be retrieved and directed toward an alternative direction, as can be seen in FIG. 4C).

According to some embodiments, Staggered side sensors 602 and 604 are positioned along the lateral aspect of the catheter, and serve to assess the gradient of dilution from the signal detected at the catheter tip sensor 606 relative to the more proximal staggered side sensors 602 and 603, for example, staggered side sensors 602 and 604 may aid in venous catheter navigation, and in optimizing as well as standardizing the ultimate placement of the catheter for continual long-term analysis.

According to some embodiments and as disclosed above, the detected signal may be endogenously produced by the target tissue/tumor, or alternatively, may be released from the arterial catheter selectively perfusing the target tissue. Alternatively, the signal may be injected through a needle into the target tissue, allowing it to travel through the tissue and into the draining vein.

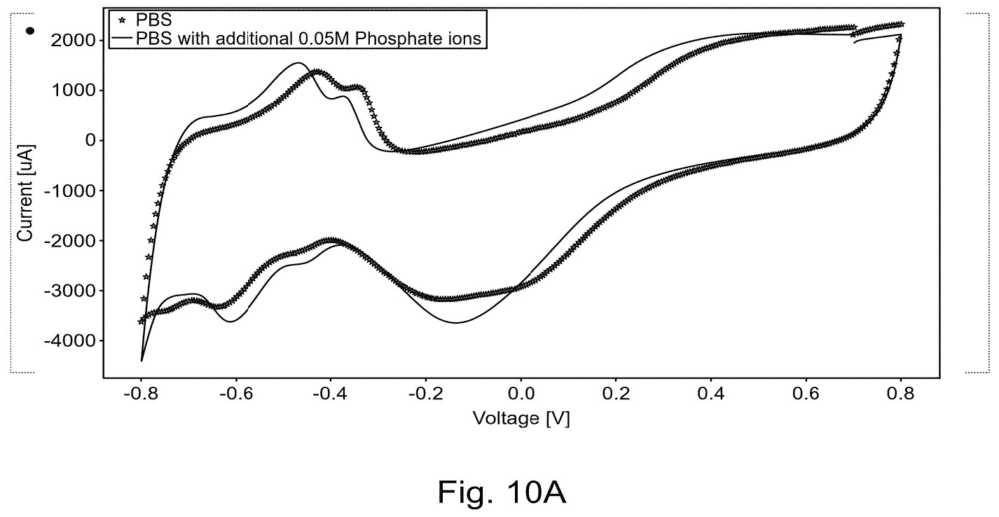

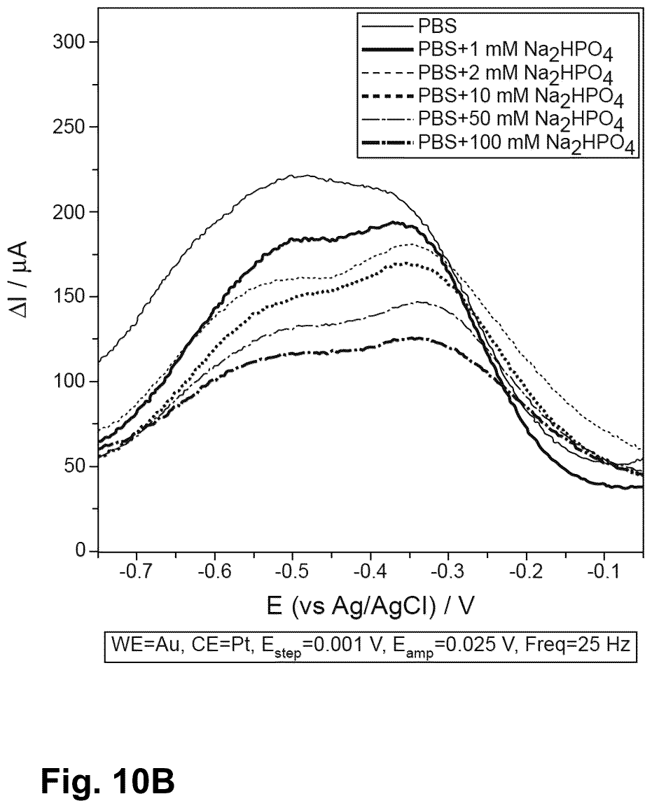

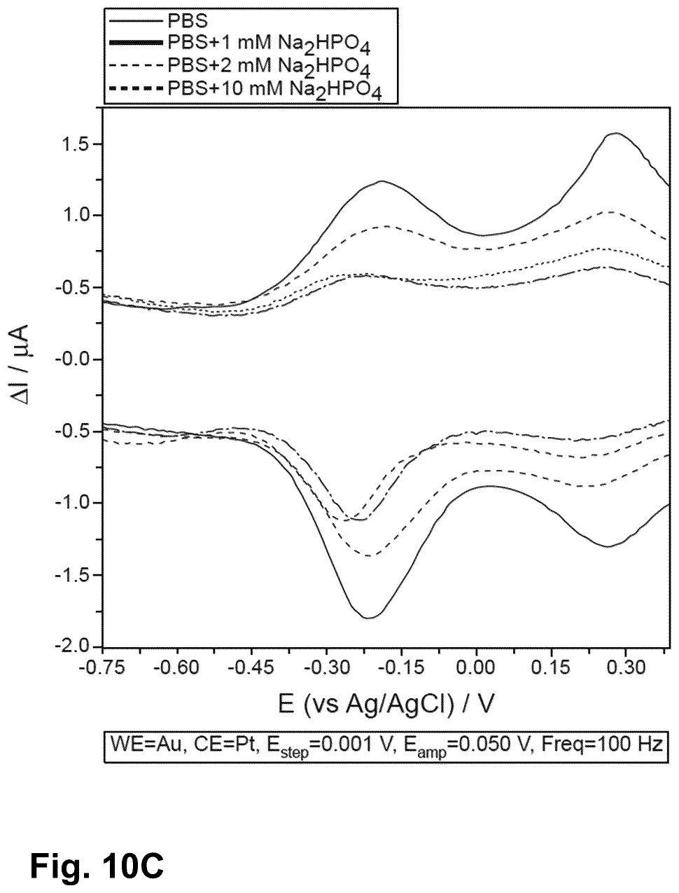

Reference is now made to FIG. 10A which graphically illustrates results of variable levels of phosphate ion concentration in a physiological fluid. As shown, an electrochemical impedance scan is conducted by using cyclic voltammetry (CV) of Phosphate Buffered Saline (PBS), which is commonly used to represent physiological and chemical properties of biological fluids.

According to some embodiments, the test results graph of two curves of a CVs performed on PBS and on PBS with a phosphate concentration higher by 5 mM than the baseline. The changes in the electric current through the linear voltage sweep are evident in the graph, and specifically, the cathodic current (negative voltage in the figure) at −0.2V is known to be characteristic of this reaction and is similar to such knowledge in the art.

According to some embodiments, in addition to the two electrodes (WE, CE) mentioned above and depicted in the graph, a reference electrode (RE) may be used to serve as a constant or near-constant known voltage as is common in many electrochemical measurements. One example of a RE is the use of a pseudo-reference electrode (PRE) which would typically be either one of the at least two electrodes mentioned above, or an additional electrode of similar or a different conductor, and potentially different size to increase stability, as well as with or without such additional components as a frit or a salt bridge for separation.

According to some embodiments, the description above refers to a direct current cyclic voltammetry, but is also applicable to square-wave, pulse, AC voltammetry, vibrational electrodes and more. According to some embodiments, an EIS may also be qualitatively and quantitatively used to analyze an analyte. According to some embodiments, an array of electrodes or microelectrodes may be used for tomographic results with spatial components or weights.

Reference is now made to FIGS. 10B-10C, which graphically illustrate experimental results of phosphate concentration measurement using gold working electrode and platinum counter electrode, and experimental results of phosphate concentration measurement using gold working electrode and platinum counter electrode, respectively.

According to some embodiments, FIG. 10B depicts experimental results validating the quantitative analysis of phosphate ions in a physiological representative liquid. Following the above stated embodiments, different results are obtained when square-wave voltammetry is applied when juxtaposed with cyclic voltammetry as shown in FIG. 10A. The results shown in FIG. 10B show a clearer response at relatively low phosphate concentrations above the physiological ambient. FIG. 10C further depicts results obtained in the same way with different ionic species found in the blood, and potentially representative of abnormal masses.

According to some embodiments, the diagnostic and therapeutic system 10 may further include other types of sensors and sensing capabilities to detect potential residues that serve as a proxy-measurement to the effects of cell death by apoptosis, necrosis or otherwise. Such process typically result in membrane-bound bodies such as microsomes or vesicles with cytoplasmic residue, cell-free nucleic acids, DNA or RNA fragments and other biological materials. Such substances and cell components typically have some spectroscopic signature in one of the relevant parts of the spectrum including UV, Visual or IR, and in either absorbance or reflectance of the incoming waves.

According to some embodiments, the diagnostic and therapeutic system 10 comprises components intended for spectrometry such as optical fibers, lights sources and typically gratings and sensors for analyses. In one embodiment the invention includes a lighting fiber optic, typically transmitting light from a source external to the patient, and a sensing fiber optic, typically with its sensing element externally to the patient. The optical equipment is typically configured to detect such spectrometric signatures as that of RNA or DNA fragments around 260 nm or alternatively Fourier Transform IR spectroscopy (FTIR) bands such as 1741 1/cm representative of phospholipid membranes, or other, wider spectrum parts that represent one or more materials in the blood stream. In other embodiments, the use of other spectrometry tools such as Fluorescence spectroscopy or Raman spectroscopy. For example, the use of Raman spectroscopy was suggested in-vivo and shown to be able to detect components as the extracellular matrix (ECM). In the context of the invention herein, the use of Raman spectroscopy can thus be used in embodiments to detect ECM components released from the target tissue into the draining venous vasculature.

According to some embodiments, non-linear optical effects are used to analyze and quantify components with the diagnostic catheter. One such example is the detection of the second harmonic generation component from a monochromatic source such as a laser. As collagen comprises a substantial portion of the tumor extracellular matrix (ECM), it is probable that the release of collagen degradation-related products will be detectable in the venous outflow of a tumor in response to the administration of ECM altering agents, such as collagenase. It is thus possible to use Second Harmonic Generation (SHG) IR, optical or UV detection by pulsating a laser source, typically at the femtosecond-range time duration, into the blood and measuring the reflected or transmitted light through a narrow-band filter designed for double the frequency of the light source. In such configurations, at least one of the optical fibers as mentioned above is connected to a photomultiplier that is typically located externally to the patient's body. The counted light emission in the relevant frequency can thus be correlated with the amount of Collagen in the blood stream. As one example, the use of Near-IR excitation lasers can be used to provide a relatively transparent part of the spectrum in blood, and to measure IR or optical emissions.

According to some embodiments, higher order frequency generation such as Third Harmonic Generation (THG) can be used to quantify components such as lipids phospholipid membranes and others. For such a purpose, filters can be applied on the emission or receiving optics as well as polarization filters.

According to some embodiments, other analytical chemistry techniques may be applied such as Nuclear Magnetic Resonance (NMR, or MRI) in conjunction with external magnetic manipulation, Electron Paramagnetic Resonance (EPR) for the detection of free radicals leaving a tumor as a result of a therapeutical payload, etc.

According to some embodiments, the analytical chemistry techniques may be applied under the effect of an external strong magnetic field and with excitation and emissions in the radio-frequency range. Furthermore, optical methods for chirality studying may be applied such as Circular Dichroism (CD) or other optical methods using light polarization. Further applicable techniques include Raman spectroscopy, tip-enhanced Raman spectroscopy, anti-Stokes Raman spectroscopy, X-ray fluorescence, typically with an external source, etc.

According to some embodiments, the use of multiple light sources and potentially of multiple readout points can be utilized for multi-or hyper-spectral imaging, typically referring to multiple spatial pixels, wherein each point contains a variety of spectral information regarding lighting for excitation and absorbance, transmittance or reflectance with the potential for non-linear effects such as fluorescence or harmonic generation.

According to some embodiments, since the chemical and physiological reaction involve by definition a variety of biological mechanisms, it is possible to include biochemical and biological detection tools in the described diagnostic catheter. Such tools can include for example Enzyme Linked Immunosorbent Assays (ELISA) can be applied to detect specific types of proteins, and specifically proteins that relate to cell death in apoptosis or alternatively in necrosis. For example, applying a surface with affinity to such proteins as caspase-cleaved Keratin 18 (ccK18), similar to commercial assays that are typically called M30 or M65 can allow the detection of apoptosis protein residue in the blood. The detection can be for example based on color by using optical fibers as described above.

According to some embodiments, the blood flow is used, or additional flow is induced to analyze a sample volume through cytometry, meaning the introduction of some external excitation and detection such as fluorescence over the flowing fluid and biological components therein. In the embodiments, implementations of flow cytometry (FC) can be included with such means as fluorescent based sorting (FACS), or cytometry channels and sensors configured for detecting specific proteins, protein activity, total DNA quantity, total RNA quantity etc.

According to some embodiments, other biological tools can be utilized to detect specific proteins, lipids concentrations or cell organelles. Technologies from the fields of ‘lab-on-a-chip’ or ‘micro-total-analytic-solutions’ (uTAS) can be intergraded on the diagnostic catheter to provide capabilities such as PCR, isothermal amplification, gel electrophoresis, liquid chromatography (LC) or others.

According to some embodiments, substances in the blood can further be characterized in other embodiments using a photoacoustic (PAS) component integrated to optically excite tissue or a blood volume and to measure acoustic and ultrasonic reaction to it. Such PAS techniques can be used independently or in conjunction with other laser excitation techniques as mentioned above, and among others with harmonic generation measurement (SHG, THG etc.). Furthermore, tomographic high resolution measurement can be performed by phase information of reflected light such as in Optical Coherence Tomography systems (OCT).

According to some embodiments, the analytical techniques described above can be further utilized to detect signals pertaining directly to the therapeutic payload rather than the biological effects. Specifically, the detection of the therapeutic agent can be performed directly as a measurement of its effective chemical spectroscopic signal. For example, Doxorubicin, a commonly used chemotherapeutical agent is fluorescent with an excitation around 500 nm and emission around 600 nm. Furthermore, Doxorubicin fluorescence is dependent on the medium it is in, and more specifically, the emission spectra may be affected by the surrounding pH or by quenching form reaction with other molecules. These all allow for gathering information through the diagnostic catheter and serve as an example of additional data that can be utilized for therapeutic monitoring and optimization.

According to some embodiments, the therapeutic payload can thus be detected to deduce whether it reached the tumor, interacted with it, or passed through ineffectively. For this purpose, direct detection using a dedicated sensor or indirect deduction using some spectrometric method can be used.

According to some embodiments, an additional material may be added through the therapeutic catheter for the purpose of detection and analysis, beyond the therapeutic payload itself. Such materials can be intended to be detected by any of the abovementioned analytical techniques or by additional techniques.

According to some embodiments, the utilization of the addition of Super-Paramagnetic Iron Oxide Nanoparticles (SPIONs) to the therapeutic payload. SPIONs can be detected by non-invasive and minimally invasive such as MRI scans or Magnetic Particle Imaging (MPI) scans as well as Magnetic Particle Spectrometry (MPS) measurements. In such embodiment nanoparticles may be added to track the arterially incoming or venously outgoing therapeutic payload. These can further be used in conjunction with biological molecules or components to react with locally found materials. For example, nanoparticles connected with antibodies can be used to detect and spatially resolve the location of such proteins as abovementioned ccK18, or alternatively proteins of the Bcl-2 family, representing and potentially quantifying the prevalence of induced apoptosis. This technique can further be used to non-invasively monitor the motion of extracellular matrix material or lipid residues such as disconnected membrane blebs.

According to some embodiments, the added material is a contrast agent configured to be visible in either x-ray imaging modalities or ultrasound imaging modalities. For example, typical x-ray contrast agents such as iodine or barium when used in conjunction with some adhering mechanism to the therapeutic payload or to a biological component for reaction with an expected outgoing material as mentioned above. In another embodiment the use of bubbles, gas-filled silica beads or other implementation of non-miscible gas that can be safely introduced for the purpose of use as an ultrasound imaging contrast agent.

According to some embodiments, the added substances or the therapeutic payload further respond with incoming external radiation such as x-rays or ultrasound via mechanisms such as scintillation, fluorescence, sonoluminescence or others.

It is further stated that some of the abovementioned techniques can benefit from concepts in quantum metrology such as, yet not limited to, photon entanglement in excitation light in such measurements as fluorescence sensing, that may benefit from measurements at low photon numbers, working around bleaching or exceeding beyond photon shot noise.

The information gathered via the diagnostic and therapeutic system 10 described above may refer to signals and comprises off quantitative series of values that are collected in the time or frequency domains. The signals may be analyzed by a variety of means that include some analog preprocessing or filtering, digital acquisition, digital filtering and others. The signals may be analyzed with one or more mathematical techniques to perform dimensionality reduction, classification, anomaly detection, or heuristic algorithms such as thresholding, peak detection, frequency domain analysis, Laplace transform analysis and others. For these purposes, such common techniques can be applied as singular value decomposition (SVD), support vector machines (SVM), clustering, gaussian mixture models, regression, artificial neural networks (ANN) and others.

According to some embodiments, the diagnostic and therapeutic system 10 may be applicative in multiple medical fields for the treatment of human patients, veterinary applications and research ones.

It is however useful to note that ongoing monitoring of the physiological and chemical effects of a therapeutic payload can allow tuning many parameters that include, but are not limited to: the specific materials that are introduced as part of the payload, the local temperature, the local pH, additional materials such as enzymes for the dissolution of specific materials, the pace at which the payload is introduced, the dose at which it is introduced, or others.

According to some embodiments, the diagnostic and therapeutic system 10 may be regarded as a tool for the medical diagnosis, information acquisition and therapeutic optimization, that can be used before, during to or in close temporal proximity to the use of some medical treatment. The main action served by the diagnostic catheter is thus referred to herein as ‘dynamic vascular biopsy’.

According to some embodiments, the diagnostic and therapeutic system 10 includes an endo-vascular system typically including a catheter selectively positionable in the arterial supply to a (suspected) diseased target tissue and a second diagnostic catheter positionable in a vein draining the target tissue (for example, by utilizing arterial catheter 102). The purpose of the arterial catheter is to provide controlled regional delivery of drugs and other agents to modify the environment of the tissue. The purpose of the venous sampling catheter is to analyze or collect blood samples from the tissue in real time. The proximity of the blood samples to the target tissue increases and potentially maximizes the concentration of soluble compounds originating from within the target tissue which are indicative of processes such as cell death (apoptosis or necrosis) or cell replication (mitosis) ongoing in the selected tissue of interest.

According to some embodiments, the proximity may also provide a way to assess the presence of cell types and activities of those cells within the target tissue: for example, the presence of lymphocytes or secreted cytokines. Selective proximity to tumor venous blood may further be used to collect high concentrations of circulating tumor cells, DNA, RNA and other soluble factors including tumor secreting proteins and hormones, for further analysis.

According to some embodiments, blood may be analyzed in-vivo, in real-time, or close to it, while in some embodiments blood is collected to be analyzed externally with classical analytical equipment. The invention further shows the combination thereof to allow for tiered information to be collected during a procedure, later (e.g. minutes) in a procedure, or afterwards while a patient is recovering. It is further mentioned that blood samples may in some embodiments be analyzed externally and circulated back into the patient's body to prevent blood loss. This is especially true in the case of analytical assays requiring high quantities of blood.

According to some embodiments, the detection and characterization of biomarkers and other entities present in venous blood samples may be obtained, e.g. using a portable laboratory console outside the body, where fluid samples are analyzed. The analysis for some entities is achievable in real time, within seconds to minutes. For characterization of other entities such as DNA sequencing, samples may be assessed using, for example, known techniques. In real time assessment, the resulting data can serve to inform which agents and/or which concentrations are to be delivered into the disease target tissue using, e.g. the arterial catheter.

For example, a target tissue can be sequentially exposed to one or more potential therapeutic agents while simultaneously recording evidence of their impact, e.g. detecting evidence of tumor cell death, in near real time.

According to some embodiments, the diagnostic and therapeutic system 10 provides both diagnostic and therapeutic benefits, as once a given therapeutic modality is assessed as effective, it can be delivered locally into the target tissue to treat the disease not just assess its efficacy. Similarly, once the sensed signal of efficacy has abated, the infused material can be switched.

According to some embodiments, particularly in cancer treatment and in view of heterogeneity and clonal variations within tumors, the disclosed dynamic biopsy system enables assessing the effect of multiple lines of therapy upon a heterogeneous population of cells over a relatively short interval (minutes to hours) rather than what is commonly performed in standard systemic chemotherapy or local chemo-or immuno-arterial drug delivery, whereby therapeutic agents are assessed for efficacy usually by imaging features every 3 to 6 months.

According to some embodiments, a further application of the diagnostic and therapeutic system 10 is in improving or optimizing conditions for therapeutic efficacy within a defined anatomic territory, e.g., improving or optimizing local pH, temperature, and other modifiers levels so as to decrease interstitial tumor pressure and/or alter the extracellular matrix for example by administration of collagenase, thereby increasing efficacy of therapeutic agents. Such improvement or optimization of the tumor microenvironment for therapy may require multiple concurrent or sequential administrations, which would be possible only through a selective vascular approach as disclosed herein.

### Methods for Optimizing Treatment Precision

According to some embodiments, at least two clinical needs prevailing in managing chronic, long-term patients, for example, cancer patients are addressed by utilizing the diagnostic and therapeutic system 10. The first is the ability to choose the most effective and appropriate therapy and/or combination of therapeutics while optimizing conditions for therapeutic effect. The second involves decreasing the (systemic) toxicity and morbidity associated with the therapy itself while maintaining its therapeutic efficacy.

According to some embodiments, the diagnostic and therapeutic system 10 may provide a method for assessing treatment efficacy in a target tissue of a subject being subjected to a treatment protocol. The method comprises at least the following steps:
(i) introducing an endovascular venous sensor-enabled catheter into a vein that collects blood drained directly from the target tissue;
(ii) subjecting the venous blood samples collected from the venous implant to an external and/or internal analysis module operable to identify and/or quantify one or more predetermined components in the venous blood; and
(iii) determining the effectiveness of treatment in the target tissue based on the presence and, optionally, the quantity of the one or more predetermined components in the drained venous blood.

According to some embodiments, the disclosed method for assessing treatment efficacy to a given treatment protocol at a target tissue may be extended and utilized for improving and/or optimizing treatment precision by further including the steps of:
(iv) changing one or more components in the treatment protocol if the efficacy of treatment in the target tissue as determined in step (iii) above, is lower than a desired predetermined efficacy; and
(v) repeating steps (iii) and (iv) one or more times until a desired optimized efficacy in the target tissue is obtained.

According to some embodiments, the effectiveness of a given treatment protocol, in accordance with embodiments described herein, may be determined in near real time and even in real time, thus enabling treatment of the target tissue in a relatively short time, for example, within 1, 3, 5, 10, 15, 24, 30, 36, 48 or 72 hours.

According to some embodiments, a treatment protocol comprises systemic administration of at least one active agent to the subject.

According to some embodiments, a treatment protocol comprises local administration, e.g., arterial administration, of at least one active agent to the target tissue. According to some embodiments, the present invention includes combining both systemic and local administration of one or more active agents.

According to some embodiments, the contemplated dynamic biopsy, enabled by the diagnostic and therapeutic system 10, enables to design or tailor a treatment protocol which would precisely provide a desired therapeutic effect at a target tissue of a subject. Such a treatment protocol may be patient-specific or personalized, specifically tailored to meet the unique needs and requirements of the subject being treated.

According to some embodiments, the diagnostic and therapeutic system 10 discloses and cites a method for designing and providing an improved or optimized treatment protocol for a target tissue in a subject, the method comprising the steps of:
(i) introducing an endovascular arterial device into an artery that supplies blood directly to the target tissue;
(ii) introducing an endovascular venous sensor-enabled device into a vein that collects blood, or monitors blood, drained directly from the target tissue;
(iii) subjecting the target tissue to a local treatment protocol delivered via the endovascular arterial device;
(iv) collecting venous blood data from the venous device and subjecting them to an external and/or internal analysis module/controller operable to identify and/or quantify one or more predetermined components in the venous blood;
(v) determining the effectiveness of treatment in the target tissue based on the presence and, optionally, the quantity of the one or more predetermined components in the drained venous blood;
(vi) changing one or more components in the treatment protocol if the efficacy of treatment in the target tissue as determined in (v) is lower than a predetermined, desired (e.g., optimal) efficacy; and
(vii) repeating steps (iii) to (vi) one or more times until improved or optimized treatment precision in the target tissue is obtained and maintained.

According to some embodiments, the diagnostic and therapeutic system 10 may be designed to operate both as a system that delivers a specific local treatment protocol via a drug delivery module, while monitoring, at the same time, the local effect of the treatment and communicating efficacy assessment readouts to the drug delivery module. Based on treatment effect readouts received, one or more parameters in the treatment protocol may be adjusted, changed and/or adapted, and a modified treatment may be delivered, optionally, via the drug delivery module, or treatment may be stopped altogether for safety or other reasons.

According to some embodiments, the diagnostic and therapeutic system 10 comprises two main elements operable to work in cooperation.

According to some embodiments, the first element in the diagnostic and therapeutic system 10, is an endovascular venous internal sensor device, comprising a venous device connected to a sensor operable to identify and quantify molecules or biological entities, which are being drained from the tissue by the blood samples collecting venous device. Such molecules may indicate physiologic- and/or metabolic-properties, which are ongoing within the tissue being drained. For example, detection of, for example, cellular division, changes in genomic phenotype, and/or evidence of cell death may be achieved and quantified in near real-time. Optionally, the introduced sensor has the capacity to transfer the recorded signals to a remote device, herein termed “remote electric port”. Optionally, the sensor device is endovascularly (venously) introduced.

According to some embodiments, the second element in the diagnostic and therapeutic system 10, is an optional port, which comprises one or more reservoirs containing therapeutic and/or diagnostic material(s), collectively referred to herein as “drug delivery module”. The reservoirs are in communication with the vascular system either systematically, intravenously or via a communicating tip positioned selectively within the arterial supply to the region of therapeutic interest, also referred to herein as “an endovascular arterial device”.

According to some embodiments, the sensor is an analysis module connected to the venous device and operable to continuously or repeatedly analyze, in-or in near-real time, venous blood samples and communicate with the electric port by providing a corresponding feedback. Thus, the two elements have the ability to connect and cooperate with each other. In such a cooperative system, the information or feedback provided by the first element is transmitted to the electric port and used for adjusting, adapting and/or otherwise modifying the treatment strategy by way of, for example, changing or replacing altogether the treatment type, changing certain parameters in a treatment protocol such as timing of administration and/or dosages of one or more active agents, replacement of one or more active agents and/or the like, so as to improve or optimize therapy precision.

### EXAMPLES

Reference is now made to the following examples, which together with the above description illustrate some embodiments of the disclosure in a non-limiting fashion.

Generally, the nomenclature used herein, and the laboratory procedures utilized in the present disclosure include molecular, chemical, biochemical, and/or microbiological, and clinical procedures. Such techniques are thoroughly explained in the literature.

### Example 1: Near Real-Time Monitoring the Impact of Local Anti-Tumor Therapy

To assess treatment efficacy in a cancerous tissue of a subject undergoing a two-step, systemic sequential treatment protocol comprising infusing of chemotherapeutic agent A (e.g., Oxaliplatin), followed by infusion of chemotherapeutic agent B (e.g., Irinotecan), the effect of treatment on the levels of secreted apoptosis markers is assessed as a function of time. For this purpose, one or more venous microcatheters are placed in veins that collect blood drained directly from the target tumor. Blood samples collected every 30-60 mins are subjected to analysis by, e.g., a portable laboratory console to detect evidence of tumor cell death resulting from the systemic treatment provided. It is expected that a steep rise in apoptosis is recorded after the infusion of chemotherapeutic agent A, followed by a drop and an additional rise with a switch to the different chemotherapeutic agent B at time of, e.g., 2 hours.

### Example 2: Optimizing Local Treatment Protocol

Based on changes in cell apoptosis markers levels as a function of treatment time that were detected and quantified in Example 1 above, effectiveness of the systemic therapy at the locus of the diseased tissue is determined. If the treatment is presenting outcomes at the target tumor that accord with expected results, the systemic treatment protocol is replaced with a local treatment strategy. For this purpose, one or more arterial microcatheters are introduced into arteries that deliver fresh blood directly to the tumor site of the subject. The one or more arterial microcatheters are connected to a drug delivery module that comprises two or more reservoirs, each containing chemotherapeutic agents A, B etc. The one or more venous microcatheters are maintained in place for continuous draining and collection of blood samples from the tumor site, which are analyzed in near real time. The blood analysis results are processed and compared to predetermined treatment results. Based on this comparison, a desired treatment regimen is designed, tailored to meet the unique needs of the specific tumor and its surrounding tissues, and is communicated to the drug delivery module such that the drug delivery module can be programed to deliver the specifically designed treatment regimen. As such, the drugs A, B and more are released from each of the reservoirs at the precise timing and concentrations dictated by designed treatment regimen. In a local regimen, the drug doses are comparatively low, but their local concentration is very high, and can be in the range of from 10 times to 500 times higher than the concentration provided by systemic delivery.

The cycle of sampling drained blood for apoptosis markers and delivering chemotherapy is repeated until there is no more evidence of continued cell death.

In a particular study, optimization of local treatment of pancreatic tumor is assessed. Each patient undergoes a PET CT immediately before treatment and then one week later.

For each person, one or more arterial catheters are placed into vessels supplying the tumor. One or more venous drainage catheters are positioned.

The patients undergo a pre-treatment comprising administration of 10 mg of collagenase at the flow rate of 0.5 mg/minute. Then, a combined infusion of paclitaxel, gemcitabine and fluorouracil (a standard combination for this type of tumor) is administered. Each drug is infused over 1 hour, and an apoptosis signal is recorded from the draining venous blood. Early effects of cell death are noted for paclitaxel and fluorouracil. The highest signal of successful apoptosis is demonstrated with gemcitabine, which is the only drug to demonstrate sustained effects after one hour. Therefore, gemcitabine is then administered for another 45 minutes, until apoptosis signals reach zero. At this point, another round of pre-treatment with collagenase is deployed. Liposomal irinotecan is infused over the course of one hour, demonstrating a moderate apoptosis signal, which dropped to zero after 40 minutes.

The entire cycle is repeated one more time, this time demonstrating no evidence of continued cell death. Tumor eradication is confirmed by PET CT.

According to some embodiments, the experiment described in these examples or in others can also be applied to animal trials, known as pre-clinical trials. In such cases, at least one type of animal, such as porcine, murine, ovine, canine, simian or other, is being subject to a treatment protocol as a specimen, with spontaneous, induced or implanted cancer model or another diseases model.

According to some embodiments, the method and system described above, is collectively and interchangeably herein referred to as “endovascular dynamic biopsy technology” or simply “technology”. In this case study, there is both diagnostic and therapeutic benefits in applying the dynamic endovascular biopsy technology as, once a given therapy is assessed as effective, it can be designed as local treatment protocol and delivered locally into the target tissue to treat the disease, not just assess its sensitivity to treatment.

### Example 3 Optimizing Conditions for Local Therapeutic Efficacy

The disclosed endovascular dynamic biopsy technology is applied for optimizing conditions for therapeutic efficacy within a defined anatomic territory, with high local concentration and effect.

For precising local conditions at a tumor site so as to enable optimized efficacy in local treatment of the cancerous tissue, a small amount of glucose is first delivered via a microcatheter into the tumor tissue, followed by delivery of a mitogenic agent that induces mitosis phase in the tumor cells and promotes DNA exposure. Then, a small dose, at high local concentration (e.g., 100 to 400 times the concentration used for systemic therapy), of a chemotherapeutic drug is delivered (via the drug delivery module and one or more arterial microcatheters), which is active predominately in the phase of mitosis, for example, a topoisonerase inhibitor such as doxorubicin. Thus, optimized efficacy in local treatment of the cancerous tissue is obtained.

In such a case, adverse effects can be induced. For example, a response of tumor lysis syndrome (TLS) can be detected via the endovascular dynamic biopsy, leading practitioners to reduce the infusion rate by e.g. 5 to 100 times.

Similarly, precision of other local conditions at a tumor site may be established prior to delivery of chemotherapy, for example, adjusting local pH and/or temperature, inducing other modifiers that decrease interstitial tumor pressure, and/or monitoring enzymes levels such as collagenase, to thereby optimize local treatment efficacy.

Although it is reasonable that such optimization will be unique for every given condition, nevertheless, it is highly likely that generalizable protocols will emerge for given diagnoses, e.g., of colon cancer, pancreatic cancer and the like etc.

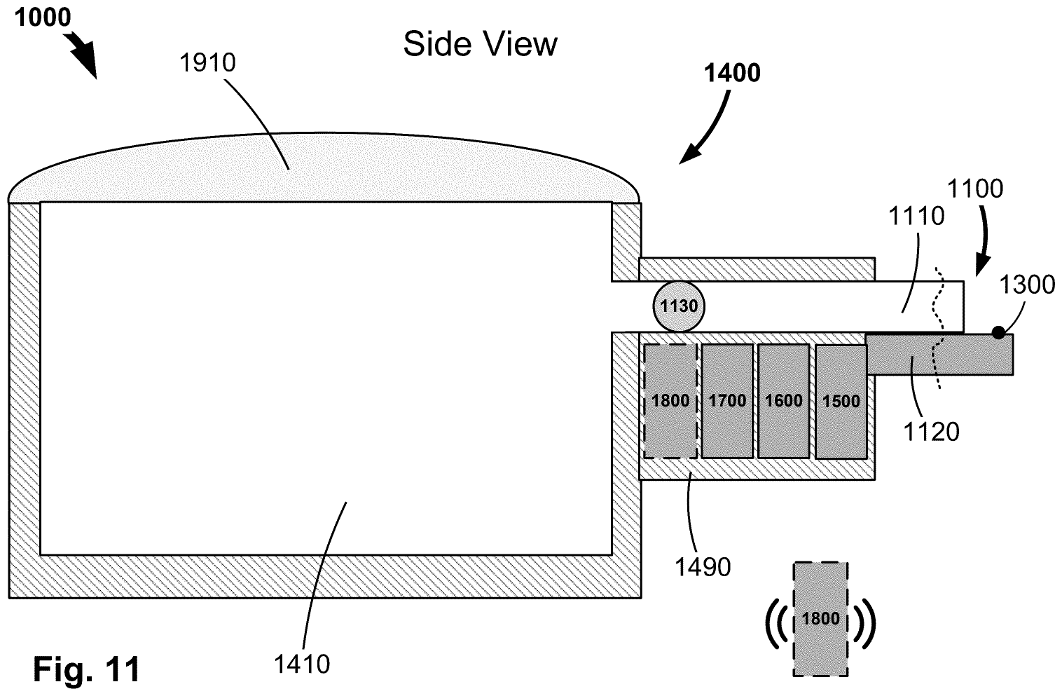

According to some embodiments, and as demonstrated in FIGS. 11, 12A, 12B, 13A, 13B and 14 a new medical system 1000 is provided, comprising:
one or more catheters 1100, 1200 each comprising at least one lumen 1110, 1120, configured to be inserted, via an endovascular invasive (EVI) device, into one or more venous vasculatures, and positioned in proximity to one or more target tissues;
one or more sensors 1300 configured to be integrated with at least one of the catheters' distal-end, and configured to monitor and/or detect biochemical- and/or physiological-changes in a venous fluid, at least partially originated from the target tissue/s, and
an implant port 1400, configured to be implanted in a tissue and to be connected to the proximal-end of the catheter/s, for communication therebetween.

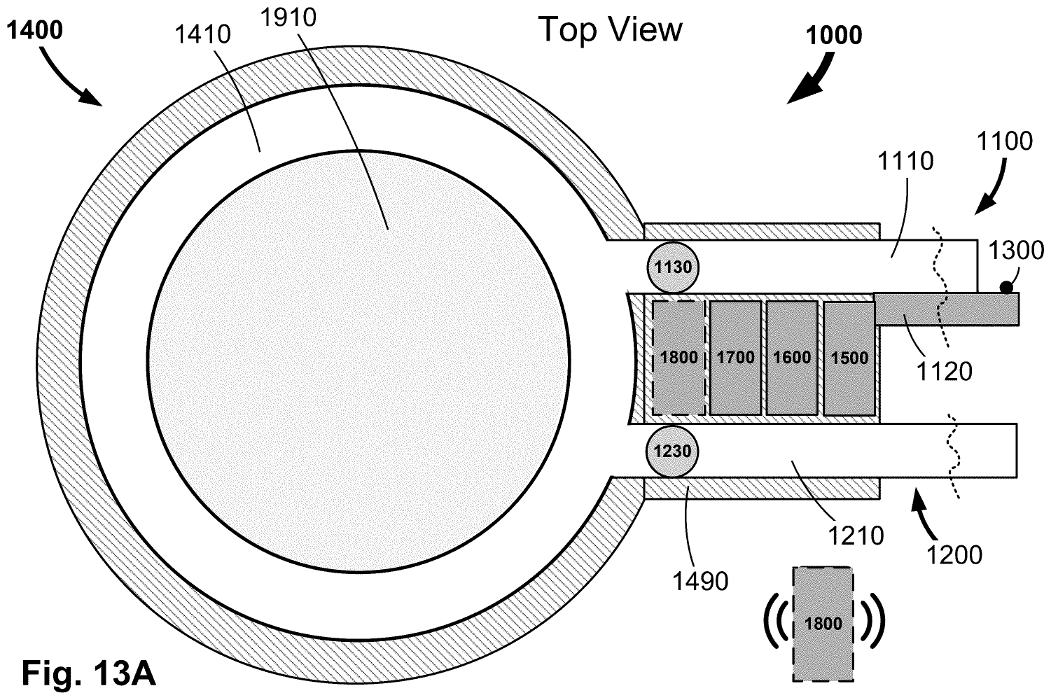

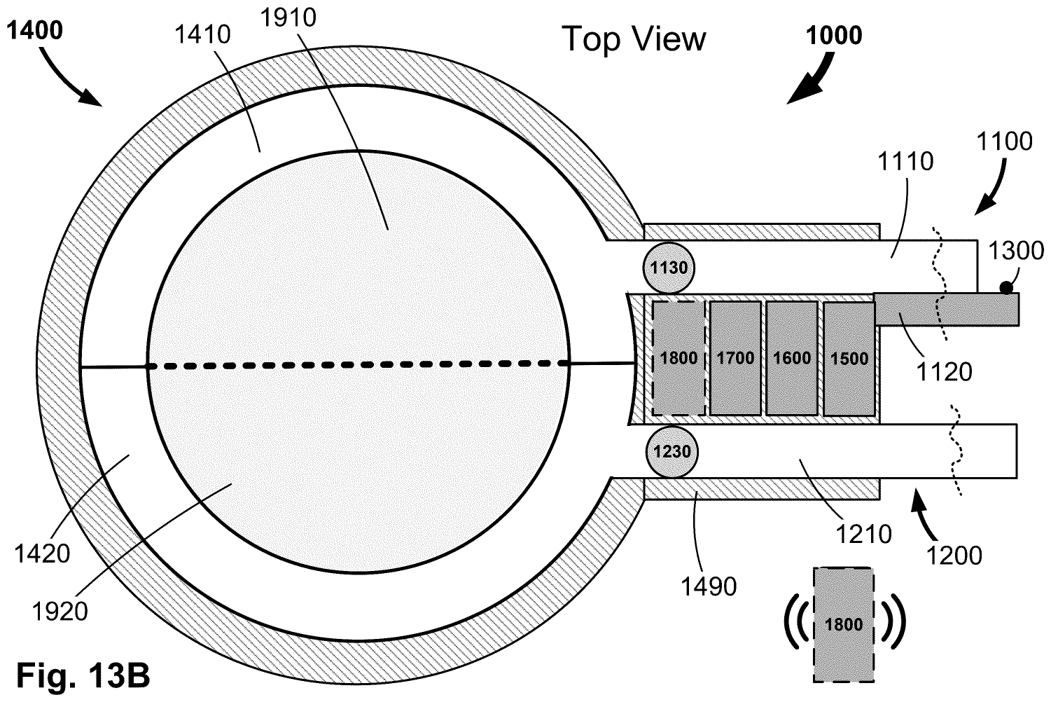

It is noted that FIGS. 11, 13A and 13B, demonstrate a diagnostic catheter 1100 having two side-by-side lumens 1110, 1120, where lumen 1110 is configured for fluid communication, and lumen 1120 is configured to accommodate the sensors 1300 and optionally their electric wires, according to some embodiments of the invention.

According to some embodiments, the port 1400 comprises:
one or more reservoirs 1410, 1420, each having an access-element 1910, 1920, and each configured to be in fluid communication with at least one of the catheters 1100, 1200;
a data-storage module 1500, configured to communicate with the sensor/s, via wired- and/or-wireless communication, and to store the monitored- and/or detected-data derived therefrom; and
a wireless receiver- and/or transmitter-module 1600, configured for wireless communication between the data-storage module and a controller 1800.

According to some embodiments, the communication with the controller can be periodic (e.g., every predetermined period of time) and/or by request/demand. According to some embodiments, the communication is configured to deliver instructions for controlling the sensors' activity.

According to some embodiments, the access-element 1910, 1920 is configured to enable exterior fluid communication with its associated reservoir 1410, 1420 and therefore with the reservoir's associated catheter/s, for at least one of:
flushing the associated catheter/s'-lumen/s and/or-sensor/s;
providing a therapeutic and/or diagnostic infusion, via the associated catheter/s; and
obtaining aspiration sample/s, via the associated catheter/s.

According to some embodiments, the exterior fluid communication can be achieved via a needle 2000, temporarily inserted into the access element, for example by a care giver and/or a machine.

According to some embodiments, the port further comprises one or more pumps 1130, 1230, configured to draw and/or push fluid between the reservoir/s 1410, 1420 and it's/their associated catheters 1100, 1200. According to some embodiments, the pumps 1130 are configured to draw and/or push fluid between the reservoir/s 1410 and it's/their associated lumens 1110, 1210.

According to some embodiments, the pump is configured to control the fluid communication between the reservoir/s and it's/their associated catheter/s and/or lumen/s, for example at least one of: draw fluid, push fluid, and block fluid communication.

According to some embodiments, and as demonstrated in FIG. 13A, the port comprises a single reservoir 1410, configured to be connected with- and in fluid communication with- at least two of the catheters 1100, 1200, and wherein:
a first catheter 1100 is configured to be positioned in proximity to the target tissue; and
a second catheter 1200 is configured to be positioned in proximity to a right atrium.

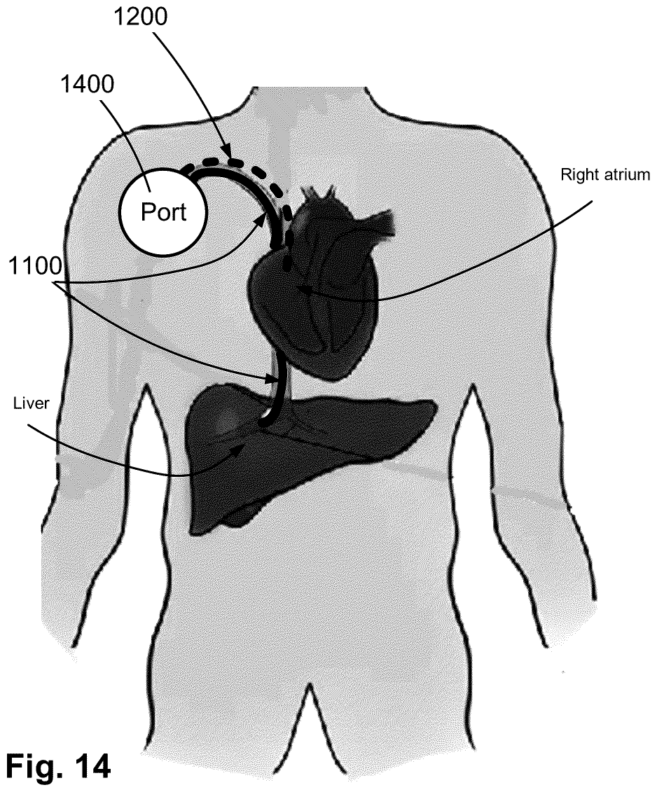

According to such embodiments, the influence of a treatment provided via the second catheter 1200 (for a non-limiting example, when administrating a systemic treatment via the right atrium (as demonstrated in FIG. 14)), can be monitored and/or detected for the target tissue, via the first catheter 1100, (for the non-limiting example, by monitoring and/or detecting biochemical- and/or physiological-changes in a venous fluid, at least partially originated from the liver's tissue (as demonstrated in FIG. 14)).

According to some embodiments, each of the catheters 1100, 1200 comprises at least one blocking-element, configured block its associated lumen/s (for example: via the pump, or via an externally controlled magnet valve (not shown)), thereby configured to enable at least one of:
blocking the lumen/s 1110 of the first catheter 1100, when pushing and/or drawing fluid via the second catheter 1200;
blocking the lumen/s 1210 of the second catheter 1200, when pushing and/or drawing fluid via the first catheter 1100;
blocking the lumens 1110, 1210 of both the first- and second-catheters 1100, 1200, to enable cleansing of the reservoir 1410.

According to some embodiments, and as demonstrated in FIG. 13B, each reservoir 1410, 1420 is configured to be connected with- and to be in fluid communication with- only one catheter 1100, 1200.

According to some embodiments, and as further demonstrated in FIG. 13B, the port comprises at least two reservoirs 1410, 1420, and wherein:
a first reservoir 1410 is configured to be connected to a first catheter 1100; the first catheter is configured to be positioned in proximity to the target tissue; and
a second reservoir 1420 is configured to be connected to a second catheter 1200; configured to be positioned in proximity to a right atrium.

According to such embodiments, the influence of a treatment provided to a target tissue, via the second reservoir 1420 and the second catheter 1200 (for a non-limiting example, when administrating treatment to the liver via the right atrium (as demonstrated in FIG. 14)), can be monitored and/or detected via the first catheter 1100, (for the non-limiting example, by monitoring and/or detecting biochemical- and/or physiological-changes in a venous fluid, at least partially originated from the liver's tissue). According to such embodiments, the first reservoir 1410 and the first catheter 1100 can be used for drawing fluid for external analysis, and/or to infusing designated substance/s having an effect on the fluid surrounding the at least one sensor 1300 in the first catheter.

According to some embodiments, each of the access-elements is differently marked, for a non-limiting example via different: colors, marks, stiches, bumps, flexibility, and the like, configured to enable a caregiver and/or a machine an identification of its associated catheter, leading to the right atrium or to the target tissue.

According to some embodiments, the implant port is a subcutaneous port, configured to be implanted under skin.

According to some embodiments, the access-element comprises a septum, configured to enable fluid to be injected to- and/or to be drawn from-the reservoir, via a needle 2000.

According to some embodiments, the port further comprises at least one power source 1700. According to some embodiments, at least one of the power sources is a battery. According to some embodiments, at least one of the power sources is a wireless rechargeable battery.

According to some embodiments, the port further comprises a controller module 1800, configured communicate via the receiver- and/or transmitter-module with other controllers 1800.

According to some embodiments, the controller module is configured to receive the monitored and/or detected data derived from the at least one sensor, to process and analyze their data and produce medical recommendations and/or conclusions.

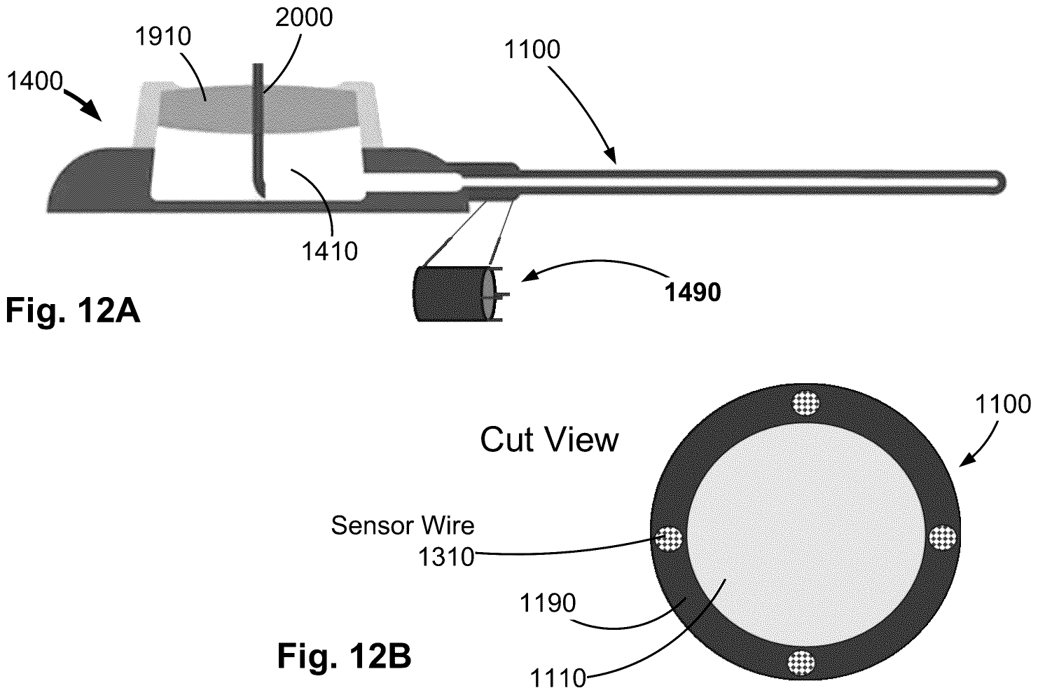

According to some embodiments, and as demonstrated in FIG. 12A, the port comprises a hub 1490, configured to enable electric data communication between the connected catheter's sensors and the port's modules.

According to some embodiments, and as demonstrated in FIGS. 12A and 12B, the diagnostic-catheter 1100 comprises a single lumen 1110 and wall layers 1190, configured to accommodate the sensors and/or their wires, wherein the external layers 1190 are configured to accommodate the sensors and/or their electric wires 1310, and wherein the lumen 1110 is configured for fluid communication.

According to some embodiments, the target tissue is a tumor and wherein the sensors/s are configured to monitor biochemical and/or physiological changes in a fluid stream at least partially originated from the tumor.

According to some embodiments, the controller further comprises visual internal control means, configured to provide spectroscopic control reference to the sensor/s.

According to some embodiments, at least two sensors are geometrically arranged in a staggered way, configured to deduce spatial and spatiotemporal information regarding biochemical and/or physiological signals, found in proximity to the device.

According to some embodiments, at least one sensor is an optical sensor configured to detect cell residues characteristic of cell death; cells are at least one of: membrane blebs, nucleic acids, DNA or RNA fragments, exosomes, nucleic fragments, structural proteins, mitochondrial proteins or other cellular debris, biochemical signals and any combination thereof.

According to some embodiments, at least one sensor is of an optical type selected from a UV-VIS spectrometer, NIR spectrometer, FTIR spectrometer, led, optical fiber and any combination thereof.

According to some embodiments, at least one sensor is configured to detect the second harmonic generation signature of Collagen.

According to some embodiments, at least one sensor is a chemical sensor configured to detect inorganic and/or organic ions.

According to some embodiments, at least one sensor is a biological sensor configured to detect cell residues characteristic of cell death.

According to some embodiments, a new medical method, comprising the steps of:
inserting at least one diagnostic-catheter, via an endovascular invasive (EVI) device, comprising at least one lumen and at least one integrated sensor, into a venous vasculature, such that the sensor/s is/are placed at a proximity to a target tissue; according to some embodiments, not all catheters have integrated sensors thereon;
implanting a subcutaneous port, the port comprising
   one or more reservoirs, each reservoir having an access-element;
   a data-storage module, configured for communicating with the sensor/s and to storing the monitored- and/or detected-data derived therefrom;
   a wireless receiver- and/or transmitter-module, configured for wireless communication between the data-storage module and a controller;
connecting the reservoir to the proximal-end of the inserted diagnostic-catheter/s for communication therebetween; each reservoir to a different catheter;
monitoring biochemical and/or physiological venous fluid at least partially originated from the target tissue, via the sensor/s, and detecting biochemical and/or physiological changes in the venous fluid, and
analyzing the monitored and/or the detected data using the controller, for producing medical recommendations and/or conclusions.

According to some embodiments, the method further comprises at least one of:
drawing fluid for external analysis (for a non-limiting example, laboratory testing), and
infusing designated substance/s having an effect on the fluid surrounding the at least one sensor;
   both via the diagnostic-catheter and a first reservoir.

According to some embodiments, the method further comprises:
inserting a therapeutic-catheter, via an EVI device, such that its distal-end is in proximity to a right atrium;
connecting a second- and different-reservoir of the subcutaneous port to proximal-end of the inserted therapeutic-catheter for communication therebetween;
infusing the right atrium with a therapeutic treatment; such that an influence of the therapeutic treatment provided to the target tissue, via the therapeutic-catheter and the second reservoir, is monitored and/or detected via the diagnostic catheter.

According to some embodiments, the steps of monitoring, detecting and analyzing of the venous fluid are provided in real-time, while the target tissue is infused.

According to some embodiments, the method further comprising removing and/or replacing sensor/s integrated on the diagnostic-catheter, via an EVI device.

According to some embodiments, the catheter system, according to any of the above mentioned embodiments, is configured to provide a more self-contained and long-term setup. The system facilitates dynamic therapy adjustments while minimizing patient discomfort and risks associated with external ports or connectors

Although the present invention has been described with reference to specific embodiments, this description is not meant to be construed in a limited sense. Various modifications of the disclosed embodiments, as well as alternative embodiments of the invention will become apparent to a person skilled in the art upon reference to the description of the invention. It is, therefore, contemplated that the appended claims will cover such modifications that fall within the scope of the invention.

1. A medical system, comprising:
one or more catheters each comprising at least one lumen, configured to be inserted, via an endovascular invasive (EVI) device, into one or more venous vasculatures, and positioned in proximity to one or more target tissues;
one or more sensors configured to be integrated with at least one of the catheters distal-end, and configured to monitor and/or detect biochemical- and/or physiological-changes in a venous fluid, at least partially originated from the target tissue/s, and
an implant port, configured to be connected to the proximal-end of the catheter/s, for communication therebetween; the port comprising:
one or more reservoirs, each having an access-element, and each configured to be in fluid communication with at least one of the catheters;
a data-storage module, configured to communicate with the sensor/s and to store the monitored- and/or detected-data derived therefrom; and
a wireless receiver- and/or transmitter-module, configured for wireless communication between the data-storage module and a controller.

2. The system of claim 1, wherein the access-element is configured to enable exterior fluid communication with its associated reservoir and therefore with the reservoir's associated catheter/s, for at least one of:
flushing the associated catheter/s'-lumen/s and/or-sensor/s;
providing a therapeutic and/or diagnostic infusion, via the associated catheter/s; and
obtaining aspiration sample/s, via the associated catheter/s.

3. The system of claim 1, wherein the port further comprises one or more pumps, configured to draw and/or push fluid between the reservoir/s and it's/their associated catheter/s.

4. The system of claim 3, wherein the pump is configured to control the fluid communication between the reservoir and it's/their associated catheter/s.

5. The system of claim 1, wherein the port comprises a single said reservoir, configured to be connected with- and in fluid communication with- at least two of the catheters, and wherein:
a first said catheter is configured to be positioned in proximity to the target tissue; and
a second said catheter is configured to be positioned in proximity to a right atrium;
such that an influence of a treatment provided to the target tissue, via the second catheter, is monitored and/or detected via the first catheter.

6. The system of claim 5, wherein each of the catheters comprises at least one blocking-element, configured block its associated lumen/s, thereby configured to enable at least one of:
blocking the lumen/s of the first catheter, when pushing and/or drawing fluid via the second catheter;
blocking the lumen/s of the second catheter, when pushing and/or drawing fluid via the first catheter; and
blocking the lumen/s of both the first- and second-catheters, to enable cleansing of the reservoir.

7. The system of claim 1, wherein each of said reservoir/s is configured to be connected with- and in fluid communication with- only one of said catheters.

8. The system of claim 7, wherein the port comprises at least two said reservoirs, and wherein:
a first said reservoir is configured to be connected to a first said catheter, configured to be positioned in proximity to the target tissue; and
a second said reservoir is configured to be connected to a second said catheter, configured to be positioned in proximity to a right atrium;
such that an influence of a treatment provided to the target tissue, via the second catheter, is monitored and/or detected via the first catheter.

9. The system of claim 8, wherein each of the access-elements is differently marked, configured to enable a caregiver an identification of its associated catheter.

10. The system of claim 1, wherein the implant port is a subcutaneous port, configured to be implanted under skin.

11. The system of claim 10, wherein the access-element comprises a septum, configured to enable fluid to be injected to- and/or to be drawn from-the reservoir, via a needle.

12. The system of claim 1, wherein the port further comprises at least one power source.

13. The system of claim 12, wherein at least one of the power sources is a wireless rechargeable battery.

14. The system of claim 1, wherein the port comprises a controller module, configured communicate via the receiver- and/or transmitter-module with other controllers.

15. The system of claim 1, wherein the controller module is configured to receive the monitored and/or detected data derived from the at least one sensor, to process and analyze their data and produce medical recommendations and/or conclusions.

16. The system of claim 1, wherein at least one of the following holds true:
the target tissue is a tumor and wherein the sensors/s are configured to monitor biochemical and/or physiological changes in a fluid stream at least partially originated from the tumor;
the controller further comprises visual internal control means, configured to provide spectroscopic control reference to the sensor/s;
at least two sensors are geometrically arranged in a staggered way, configured to deduce spatial and spatiotemporal information regarding biochemical and/or physiological signals, found in proximity to the device;
at least one sensor is an optical sensor configured to detect cell residues characteristic of cell death; cells are at least one of: membrane blebs, nucleic acids, DNA or RNA fragments, exosomes, nucleic fragments, structural proteins, mitochondrial proteins or other cellular debris, biochemical signals and any combination thereof;
at least one sensor is of an optical type selected from a UV-VIS spectrometer, NIR spectrometer, FTIR spectrometer, led, optical fiber and any combination thereof;
at least one sensor is configured to detect the second harmonic generation signature of Collagen;
at least one sensor is a chemical sensor configured to detect inorganic and/or organic ions; and
at least one sensor is a biological sensor configured to detect cell residues characteristic of cell death.

17. A medical method, comprising the steps of:
inserting at least one diagnostic-catheter, via an endovascular invasive (EVI) device, comprising at least one lumen and at least one integrated sensor, into a venous vasculature, such that the sensor/s is/are placed at a proximity to a target tissue;
implanting a subcutaneous port, comprising
one or more reservoirs, each reservoir having an access-element;
a data-storage module, configured for communicating with the sensor/s and to storing the monitored- and/or detected-data derived therefrom;
a wireless receiver- and/or transmitter-module, configured for wireless communication between the data-storage module and a controller;
connecting the reservoir/s to the proximal-end of the inserted diagnostic-catheter/s for communication therebetween; each reservoir to a different catheter;
monitoring biochemical and/or physiological venous fluid at least partially originated from the target tissue, via the sensor/s, and detecting biochemical and/or physiological changes in the venous fluid, and
analyzing the monitored and/or the detected data using the controller, for producing medical recommendations and/or conclusions.

18. The method of claim 17, further comprising at least one of:
drawing fluid for external analysis, via diagnostic-catheter and a first reservoir;
infusing designated substance/s having an effect on the fluid surrounding the at least one sensor, via diagnostic-catheter and a first reservoir; and
removing and/or replacing sensor/s integrated on diagnostic-catheter, via an EVI device.

19. The method of claim 17, further comprising at least one of the steps of:
inserting a therapeutic-catheter, via an EVI device, such that its distal-end is in proximity to a right atrium, and connecting a second- and different-reservoir of the subcutaneous port to a proximal-end of the inserted therapeutic-catheter, for communication therebetween; and
infusing the right atrium with a systematic therapeutic treatment via the therapeutic-catheter and the second reservoir, and monitoring and/or detecting an influence of the provided therapeutic treatment for a target tissue, via the diagnostic catheter.

20. The method of claim 19, wherein the steps of monitoring, detecting and analyzing of the venous fluid are provided in real-time, while the target tissue is infused.
